(19) United States
(12) Patent Application Publication
(10) Pub. No.: US 2023/0201147 A1
(43) Pub. Date: Jun. 29, 2023

(54) COCRYSTAL ANTIOXIDANTS OF PROTOCATECHUIC ACID WITH L-THEANINE FOR THE TREATMENT OF OXIDATIVE STRESS AND INFLAMMATORY CONDITIONS

(71) Applicant: THEAPRIN PHARMACEUTICALS INC., Hauppauge, NY (US)

(72) Inventors: Philip V. FELICE, Smithtown, NY (US); Harry G. BRITTAIN, Milford, NJ (US)

(73) Assignee: THEAPRIN PHARMACEUTICALS INC., Hauppauge, NY (US)

(21) Appl. No.: 18/050,734

(22) Filed: Oct. 28, 2022

Related U.S. Application Data
(63) Continuation of application No. PCT/US2021/031324, filed on May 7, 2021.
(60) Provisional application No. 63/021,931, filed on May 8, 2020.

(51) Int. Cl. A61K 31/192; A61K 31/198; A61K 9/00; A23L 33/175; A23L 2/52

(52) U.S. Cl. CPC A61K 31/192; A61K 31/198; A61K 9/0095; A23L 33/175; A23L 2/52

(57) ABSTRACT

A natural water soluble protocatechuic acid and L-theanine cocrystal composition is described. The cocrystal composition may be created by a method including solvent-assisted mechanical grinding. The natural water soluble cocrystal composition is suitable for sublingual administration, preferably to humans.

### CROSS-REFERENCE TO RELATED APPLICATIONS

The present application claims priority to U.S. Provisional Application No. 63/021,931, filed May 8, 2020, which is incorporated herein by reference in its entirety.

### FIELD OF THE INVENTION

The invention relates to a novel cocrystal formulation utilizing water-soluble natural antioxidants of protocatechuic acid with L-theanine for the treatment of oxidative stress and inflammatory conditions.

### BACKGROUND OF THE INVENTION

Natural anti-inflammatory supplements are becoming increasingly popular in light of the unfavorable side effect profiles of anti-inflammatory drugs (NSAIDs), some of which include sodium retention which may precipitate fluid retention in patients with heart failure and hypertension, increased risk of gastrointestinal bleeding, worsening of asthma, renal failure and an increased risk for major cardiovascular events (aspirin excluded). The salutary benefits that natural antioxidants offer play a significant role in maintaining optimal health. Inflammation is a defense mechanism in the body. It is the activation of the body's immune system in response to damaged tissue, pathogens and irritants. The inflammatory response pathway involves a coordinated array of several immune cells and blood vessels via a complex cascade of molecular signals. The inflammatory response has four phases: Inflammatory inducers (infection or tissue damage), inflammatory sensors (mast cells and macrophages), inflammatory mediators (cytokines, chemokines), and the tissues that are effected [1]. Inflammation can be divided into acute and chronic depending on the inflammatory response pathway involved. Acute inflammation lasts minutes to days and is characterized by exudation of fluid and plasma proteins and emigration of leukocytes, predominately neutrophils, whereas chronic inflammation is characterized by inflammation of prolonged duration (weeks or months) and is associated histologically by lymphocytes and macrophages, proliferation of blood vessels, fibrosis and necrosis [2]. Inflammation is a major factor for the progression of various chronic diseases/disorders, including diabetes, cancer, cardiovascular diseases, eye disorders, arthritis, obesity, autoimmune diseases, and inflammatory bowel disease [3]. Inflammation is the end result of oxidative stress. In normal and healthy body condition, there is a balance between reactive oxygen species formation/free radical and endogenous antioxidant defense mechanisms [3]. However, if this equilibrium is disturbed, it can lead to oxidative stress and associated damage [3]. This oxidative stress condition can cause injury to all vital cellular components such as DNA, proteins, and membrane lipids and it may lead to cell death [3,4]. Free radicals such as superoxide (O₂⁻) anions are produced as by-products of oxygen metabolism during oxidative phosphorylation. These radicals (O₂⁻) are formed when molecular oxygen gains an extra electron (reduction) from the electron transport chain in the mitochondria. Superoxide dismutase is a powerful antioxidant enzyme in the body that catalyzes the dismutation (simultaneous oxidation and reduction) of the superoxide radical (O₂⁻) into molecular oxygen or hydrogen peroxide (H₂O₂). Intracellular H₂O₂ is then actively degraded by catalase into oxygen and water. Antioxidant enzymes such as glutathione peroxidase, glutathione reductase, catalase and superoxide dismutase assists the body in replacing cells that are damaged by free radicals. As such, antioxidant enzymes play a critical role in the body's defense against excessive formation of O₂⁻ radicals thereby reducing oxidative damage and inflammation, and subsequently restoring intracellular redox homeostasis.

Oxidative stress can result from xenobiotics such as environmental pollutants such as sulfur dioxide, nitrogen dioxide, ozone (O₃), carbon monoxide, petroleum hydrocarbons, particulate matter (fine particles 2.5 μm in diameter or less) resulting from combustion related to motor vehicles, residential wood burning stoves, forest fires, and power plants and coarse particles (2.5-10 μm in diameter) resulting from dust stirred up by vehicles on roads and crushing or grinding operations [5]; radioactive contamination of the soil such as alpha emitters and actinide in the environment and non-actinides such as radon and radium; non-ionization radiation (ultraviolet and infrared); ionizing radiation (X rays, gamma rays), the aurora borealis (Northern Lights) is an example of charged particles from a proton storm that emits ionizing radiation; environmental noise from aircraft, especially jets breaking the sound barrier, construction sites, and air conditioning systems; chemicals such as pesticides, herbicides, polychlorinated biphenyls, cleaning products, glues; heavy metals, carcinogens, hypoxia, physical trauma, emotional stress, and nutritional.

### SUMMARY OF THE INVENTION

In one embodiment, the disclosure relates to a composition comprising protocatechuic acid and L-theanine. In one embodiment, protocatechuic acid and L-theanine are in the form of a cocrystal. In one embodiment, the wt. % of protocatechuic acid in the composition is between about 40% and about 50%. In one embodiment, the wt. % of protocatechuic acid in the composition is selected from the group consisting of about 46.4%, about 46.8%, and about 46.9%. In one embodiment, the wt. % of L-theanine in the composition is between about 50% and about 60%. In one embodiment, the wt. % of L-theanine in the composition is selected from the group consisting of about 53.1%, about 53.2%, and about 53.6%.

In one embodiment, the disclosure relates to a dosage form comprising a composition comprising protocatechuic acid and L-theanine. In one embodiment, protocatechuic acid and L-theanine are in the form of a cocrystal. In one embodiment, the dosage form is an oral disintegrating tablet or an amount of powder. In one embodiment, the amount of protocatechuic acid in the dosage form is between about 300 mg and about 450 mg. In one embodiment, the amount of protocatechuic acid in the dosage form is selected from the group consisting of about 301 mg, about 306 mg, and about 441 mg. In one embodiment, the amount of L-theanine in the dosage form is between about 300 mg and about 550 mg. In one embodiment, the amount of L-theanine in the dosage form is selected from the group consisting of about 342 mg, about 347 mg, and about 504 mg.

In one embodiment, the disclosure relates to a cocrystal protocatechuic acid and L-theanine formulation, wherein the formulation can bypass the hepatic first pass effect, with enhanced dissolution rate and bioavailability when a rapid onset of action is desired.

In one embodiment, the disclosure relates to a cocrystal protocatechuic acid and L-theanine formulation, wherein the formulation is water soluble and crosses the blood-brain barrier.

In one embodiment, the disclosure relates to cocrystal compositions of a drug from a specified drug class, and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R₁-group [C₄ or gamma-CH₂—C(O)—NR1] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In one embodiment, the disclosure relates to a method of treating a disease or disorder in a subject in need thereof. In one embodiment, the method includes administering to the subject a composition of the invention. In one embodiment, the disease or disorder is selected from the group consisting of an acute inflammatory disease, a chronic inflammatory disease, a cardiovascular inflammatory disease, oxidative stress, diabetes, diabetic retinopathy, a hypercoagulable disorder, apoptosis, and a neurodegenerative disease. In one embodiment, the protocatechuic acid and L-theanine cocrystal are absorbed in the subject's bloodstream via the sublingual route and bypass the hepatic first pass effect.

In one embodiment, the disclosure relates to a method of treating a disease or disorder in a subject in need thereof, comprising administering to the subject a dosage form of the invention. In one embodiment, the disease or disorder is selected from the group consisting of an acute inflammatory disease, a chronic inflammatory disease, a cardiovascular inflammatory disease, oxidative stress, diabetes, diabetic retinopathy, a hypercoagulable disorder, apoptosis, and a neurodegenerative disease. In one embodiment, the protocatechuic acid and L-theanine cocrystal are absorbed in the subject's bloodstream via the sublingual route and bypass the hepatic first pass effect.

In one embodiment, the disclosure relates to a method of manufacturing a composition comprising protocatechuic acid and L-theanine. In one embodiment, the method includes grinding a mixture containing protocatechuic acid and L-theanine and an alcohol solution in an agate mortar. In one embodiment, the alcohol solution is 70% isopropanol. In one embodiment, the grinding is repeated until the mixture is dry. In one embodiment, the mixture forms a slurry after grinding.

In some embodiments, the invention relates to one or more of the following cocrystal products:

| | |
|---|---|
| Cocrystal#1 | |
| Protocatechuic Acid | 441 mg (46.4%) |
| Theanine | 504 mg (53.6%) |
| Cocrystal#2 | |
| Protocatechuic Acid | 301 mg (46.8%) |
| Theanine | 342 mg (53.2%) |
| Cocrystal#3 | |
| Protocatechuic Acid | 306 mg (46.9%) |
| Theanine | 347 mg (53.1%) |

### BRIEF DESCRIPTION OF DRAWINGS

FIG. 1A illustrates an X-ray powder diffraction pattern of the L-theanine cocrystal product with 2-hydroxy-benzoic acid.

FIG. 1B illustrates a Differential scanning calorimetry thermogram of the L-theanine cocrystal product with 2-hydroxy-benzoic acid.

FIG. 2A illustrates an X-ray powder diffraction pattern of the L-theanine cocrystal product with 3-hydroxy-benzoic acid.

FIG. 2B illustrates a differential scanning calorimetry thermogram of the L-theanine cocrystal product with 3-hydroxy-benzoic acid.

FIG. 3A illustrates an X-ray powder diffraction pattern of the L-theanine cocrystal product with 4-hydroxy-benzoic acid.

FIG. 3B illustrates a differential scanning calorimetry thermogram of the L-theanine cocrystal product with 4-hydroxy-benzoic acid.

FIG. 4A illustrates an X-ray powder diffraction pattern of the L-theanine cocrystal product with 2,3 -dihydroxy-benzoic acid.

FIG. 4B illustrates a differential scanning calorimetry thermogram of the L-theanine cocrystal product with 2,3-dihydroxy-benzoic acid.

FIG. 5A illustrates an X-ray powder diffraction pattern of the L-theanine cocrystal product with 2,4-dihydroxy-benzoic acid.

FIG. 5B illustrates a differential scanning calorimetry thermogram of the L-theanine cocrystal product with 2,4-dihydroxy-benzoic acid.

FIG. 6A illustrates an X-ray powder diffraction pattern of the L-theanine cocrystal product with 2,5-dihydroxy-benzoic acid.

FIG. 6B illustrates a differential scanning calorimetry thermogram of the L-theanine cocrystal product with 2,5-dihydroxy-benzoic acid.

FIG. 7A illustrates an X-ray powder diffraction pattern of the L-theanine cocrystal product with 3,4-dihydroxy-benzoic acid.

FIG. 7B illustrates a differential scanning calorimetry thermogram of the L-theanine cocrystal product with 3,4-dihydroxy-benzoic acid.

FIG. 8A illustrates an X-ray powder diffraction pattern of the L-theanine cocrystal product with 3,5 -dihydroxy-benzoic acid.

FIG. 8B illustrates a differential scanning calorimetry thermogram of the L-theanine cocrystal product with 3,5-dihydroxy-benzoic acid.

FIG. 9A illustrates an X-ray powder diffraction pattern of an L-theanine/protocatechuic acid cocrystal product associated with the solubility study.

FIG. 9B illustrates an X-ray powder diffraction pattern of a second L-theanine/protocatechuic acid cocrystal product associated with the solubility study.

### DETAILED DESCRIPTION

There is a clear unmet need for a method for synthesizing a soluble crystal formed by protocatechuic acid and l-theanine which is readily administerable to individuals through a variety of media. The present invention satisfies these and other needs and overcomes deficiencies found in the prior art.

Role of Antioxidants in Inflammation
Free Radicals: A free radical is a molecule or atom that carries one or more unpaired electrons and is able to exist independently [3,6]. Meanwhile, free radicals have an odd number of electrons; this makes them short lived, highly reactive, and unstable [3]. Consequently, it can react quickly with other substances trying to catch the required electron to obtain stability [3]. Free radicals can become balanced by attacking the closest stable molecule and “stealing” its electron. Meanwhile the attacked molecule can become a free radical by losing its electron and start a chain reaction cascade causing damage to the living cell [3,7]. Examples of free radicals are hydroxyl free radical, superoxide free radical anion, lipid peroxyl, lipid peroxide, and lipid alkoxyl. Reactive oxygen species (ROS) are radical derivatives such as singlet oxygen and hydrogen peroxide (Table 1) [3,6,7]. Singlet oxygen is an excited or higher energy form of oxygen where the spin of a pair of electrons is in opposite directions, compared to the electron spin of normal molecular oxygen which is unidirectional.

TABLE 1
List of free radicals and their reactivity

| | Free | |
|---|---|---|
| No. | Radicals | Reactivity |
| 1 | Superoxide | Generated in |
| | anion | mitochondria, |
| | | cardiovascular |
| | | system, and |
| | | other cell types |
| 2 | Hydrogen | Formed in the |
| | peroxide | human body by a |
| | | large number of |
| | | reactions and |
| | | yields potent |
| | | reactive species |
| 3 | Hydroxyl | Highly reactive |
| | radical | and generated |
| | | during iron |
| | | overload and |
| | | such conditions |
| | | in the human |
| | | body |
| 4 | Peroxyl | Reactive and |
| | radical | formed from |
| | | lipids, proteins, |
| | | DNA, and sugar |
| | | molecules during |
| | | oxidative |
| | | damage |
| 5 | Nitric oxide | Neurotransmitter |
| | | and blood |
| | | pressure |
| | | regulation and |
| | | can yield potent |
| | | oxidants during |
| | | pathological |
| | | states |
| 6 | Peroxynitrite | Highly reactive |
| | | and formed from |
| | | NO and |
| | | superoxide |
| 7 | Ozone | Present as an |
| | | atmospheric |
| | | pollutant and can |
| | | react with |
| | | various |
| | | molecules |

Normal cellular metabolism produces ROS and these play crucial roles in activation of signaling pathways in animal and plant cells which alter the intra- and extracellular metabolism. Almost most of the ROS are produced in cells through the mitochondrial respiratory chain [3,6,7]. During endogenous metabolic reactions, aerobic cells generate ROS (e.g., superoxide anion, hydrogen peroxide (H₂O₂), and hydroxyl radical and organic peroxides) as the usual products of biological diminution of molecular oxygen [3]. Within hypoxic situation, the mitochondrial respiratory chain also generates nitric oxide (NO), which can produce other reactive nitrogen species (RNS) [3,6]. RNS can produce other additional reactive species, for example, reactive aldehydes-malondialdehyde and 4-hydroxynonenal, by inducing excessive lipid peroxidation [3]. Lipids and proteins are important targets for oxidative attack and alteration of these molecules can enhance the mutagenesis process [3,8,9].

In inflammatory response, leukocytes and mast cells are present in the damage regions which direct to a “respiratory burst” as a result of an enhanced uptake of oxygen and therefore enhance the production and release of ROS at the damaged area [3,8,9]. However, inflammatory cells generate more soluble inflammatory mediators such as cytokines, arachidonic acid, and chemokines, which act through active inflammatory cells in the area of infection and release more reactive species [3]. These essential markers can stimulate signal transduction cascades in addition to alterations in transcription factors, like nuclear factor kappa B (NF-κB), signal transducer and activator of transcription 3, activator protein-1, NF-E2 related factor-2, nuclear factor of activated T cells, and hypoxia-inducible factor-1α (HIF1-α), which mediate vital cellular stress reactions. Initiation of cyclooxygenase-2 (COX-2), inducibility of nitric oxide synthase (iNOS), and high expression of inflammatory cytokines, including tumor necrosis factor-α (TNF-α), interleukin-1β (IL-1β), IL-6, and chemokines (CXC chemokine receptor 4), in addition to changes in the expression of specific microRNAs, have also been exhibited to have a role in oxidative stress-induced inflammation [3,10,11]. This inflammatory/oxidative environment triggers an unhealthy circle, which can harm healthy stromal cells and neighboring epithelial cells, which after a long period of time may trigger carcinogenesis [3,7,10].

Some factors that cause inflammation include [12]:
oxidative stress, due to decreased levels of antioxidants, in particular vitamin D, glutathione and superoxide dismutase
an increase in sugar and foods which convert to glucose
decreased selenium levels, selenium is needed for the production of glutathione
increased estrogen levels (estrogen is an excitatory, inflammatory hormone).
decreased zinc levels and increased copper levels.
high levels of matrix metalloproteinases (MMPs). These enzymes break down protein causing a pathological reaction such as inflammation and tissue degeneration when in excess.
excess estrogen (stimulates matrix metalloproteinases).
a lack of progesterone (Progesterone suppresses MMPs and estrogen).
a predominance of the inflammatory Th1 cytokines over the anti-inflammatory Th2 cytokines.
high levels of advanced glycation endproducts (AGEs).
high levels of tumor necrosis factor-alpha and interleukin-6.
high levels of prolactin (inflammatory hormone).
increased levels of fructosamine.
increased levels of homocysteine.
high levels of pro-inflammatory HDLs, leading to increased lipid peroxidation.
decreased levels of vitamins B2, C, E and beta-carotene.

Isoprostanes and high levels of malondialdehyde, both markers for oxidative stress, are formed when fats are oxidized [12].

The antioxidant activity of hydroxybenzoic acids depends on the number of hydroxyl groups in a molecule [13,14] and it increases following the order: monohydroxy, dihydroxy, and trihydroxy, respectfully [13,15]. The antioxidant ability of the phenolic acids is greatly influenced by the number and the relative position of the hydroxyl groups in the ring [13]. The proximity of the hydroxyl groups to the acid moiety promotes the hydrogen atom transfer from the phenolic acid (PhO—H) to the radical specie [13]. In the radical scavenging mechanisms, the reactive radical specie is inactivated by accepting a hydrogen atom from a hydroxyl group of the phenolic acid [13]. Phenolic acids can scavenge free radicals through three competitive mechanisms: hydrogen atom transfer, single-electron transfer followed by proton transfer, and sequential proton loss electron transfer [13,16-23].

Recently, it has been recognized that many substances may cocrystallize in a single continuous lattice structure, leading pharmaceutical scientists into new areas of crystal engineering. Cocrystals are mixed crystals where the cocrystal is a structurally homogeneous crystalline material that has been formed from discrete neutral molecular species that are solids at ambient temperatures. Cocrystals are characterized by two or more molecules that associate but do not bond on the molecular level. Cocrystal engineering may be used to improve one or more physical properties such as solubility, stability, and dissolution rate of the active pharmaceutical ingredient of selected treatment or prevention.

One could consider cocrystals as being an alternative to polymorphs, solvatomorphs, and salts, as cocrystals represent a different approach to solve problems related to dissolution, crystallinity, and hygroscopicity. Cocrystals are attractive to the pharmaceutical industry because they offer opportunities to modify the chemical and/or physical properties of an API without the need to make or break covalent bonds. Unfortunately, it is not yet possible to predict whether two substances will cocrystallize or not, and therefore cocrystal screening studies are largely empirical in nature.

Theanine is found in green tea leaves Camellia sinensis and in the non-edible mushroom Xerocomus badius [24]. Theanine is synthesized in the root of the tea plant and concentrates in the leaves, where sunlight converts theanine into polyphenols [24]. L-theanine (N-ethyl-L-glutamine) an amino acid analog of glutamine, is a non-protein amino acid. Being 5-N-ethyl glutamine, theanine differs from glutamine by the CH₂-CH₃ (ethyl) group replacing hydrogen. The N-ethyl group confers on theanine its active properties. Theanine is hydrolyzed in the kidney to glutamic acid and ethylamine by the enzyme glutaminase [25].

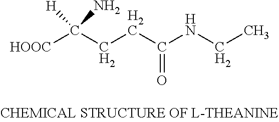

L-Theanine is an odorless, white crystalline powder that is soluble in water and transparent in solution. L-theanine has a Chemical Abstracts Service (CAS) Registry Number of 3081-61-6 and a GRAS classification (GRAS Notice Number: GRN 000209). L-theanine has the molecular formula C₇H₁₄N₂O₃, molecular weight of 174.20 g/mol, pKa of 2.35,a melting point of 217-218° C. and has an LD₅₀ of greater than 5000 mg/kg in rats [24].

Anti-inflammatory properties of L-Theanine: In various animal studies, L-Theanine's anti-inflammatory properties were shown to inhibit the expression of several inflammatory factors including IL-1β, TNF-α, IL-6, inhibit the expression of pro-inflammatory mediators involved in the nuclear factor-kappa B pathway, such as inducible nitric oxide synthase (iNOS) and matrix metalloproteinase-3, suppress the acute phase response of C-reactive protein levels [26,27], promote the expression of the anti-inflammatory cytokine IL-10 [27], and to inhibit pro-inflammatory PKC/ERK/ICAM-1/IL-33 signaling [28].

Antifibrotic properties of L-Theanine: L-theanine's antifibrotic properties were shown in one animal study to down-regulate the profibrotic cytokines TGF-β, CTGF and promote the expression of the fibrolytic enzyme metalloproteinase-13 [27]. Liver hydroxyproline contents and histopathological analysis demonstrated the antifibrotic effect of L-theanine [27].

Antioxidant properties of L-Theanine: L-Theanine's antioxidant properties are in part related to its role in preventing lipid oxidation of low density lipoprotein cholesterol [29]. In one animal study, L-theanine restored the antioxidant capacity of hepatocytes including glutathionine content and superoxide dismutase activity which were reduced by ethanol [30]. These results indicated that L-theanine prevented ethanol-induced liver injury through enhancing hepatocyte antioxidant abilities [30]. In another animal study, theanine decreased doxorubicin-induced adverse reactions such as the induction of the lipid peroxide level and the reduction of glutathione peroxidase activity in normal tissues [31]. Moreover, theanine inhibited glutathione (GSH) reduction induced by doxorubicin in the liver and heart [31]. These results suggested that theanine attenuated the doxorubicin induced adverse reactions involved in oxidative damage, due to increase in glutamate and the recovery of GSH in normal tissues [31].

Anti-angiogenic properties of L-Theanine: Retinal neovascularization or angiogensis is the formation of new blood vessels originating from the retinal veins and extending along the inner (vitreal) surface of the retina. Neovascularization within the eye contributes to visual loss in several ocular diseases, the most common of which are proliferative diabetic retinopathy, neovascular age-related macular degeneration, and retinopathy of prematurity [32]. L-theanine's potential effect on suppressing neovascularization via multiple signaling pathways in the rat model of oxygen-induced retinopathy was demonstrated in an animal study [33]. In that study, gastric gavage of L-theanine reduced the angiogenic cytokine VEGF-A (vascular endothelial growth factor-A), VEGFR-1 (vascular endothelial growth factor receptor-1), IGF-1(insulin-like growth factor-1), and MMP-9 (matrix metallopeptidase-9) mRNA levels compared to distilled water treatment. Although not wishing to be bound by any particular theory, these data suggest the potential utility of L-theanine for prophylactic measures in ocular disorders associated with retinal neovascularization [33]. In another animal study, the protective effect of L-theanine against neurotoxicity of retinal ganglion cells of rats with chronic moderately elevated intraocular pressure was demonstrated. Pre-treatment and early post-treatment with theanine was shown to be an effect neuroprotectant in the rat model of chronic glaucoma [34].

Cardioprotective properties of L-Theanine: L-Theanine's cardioprotective properties are multifold and related to its anti-inflammatory effect, antioxidant effect, anti-atherogenic effect, stimulation of nitric oxide production, calming effect and anticoagulant effect. Inflammation and peroxidation of lipids are thought to play a major role in atherosclerosis [27,28,3 0]. Inflammation and inflammatory cell infiltration are the hallmarks of myocardial infarction and reperfusion injury [35]. Ischemic cardiac injury activates the innate immune response via toll-like receptors and upregulates chemokine and cytokine expressions in the infarcted heart [35]. Sequential infiltration of the injured myocardium with neutrophils, monocytes and their descendant macrophages, dendritic cells, and lymphocytes contributes to the initiation and resolution of inflammation, infarct healing, angiogenesis, and ventricular remodeling [35]. L-theanine inhibits the expression of several inflammatory factors, inhibits the expression of pro-inflammatory mediators involved in the nuclear factor kappa B pathway, suppresses the acute phase response of C-reactive protein levels [26,27], promotes the expression of the anti-inflammatory cytokine IL-10 [27], and inhibits lipid peroxidation of low density lipoprotein [29]. As such, L-theanine's anti-atherogenic effect is secondary to its anti-inflammatory activity and peroxidation inhibition. L-theanine promotes nitric oxide production in endothelial cells through eNOS phosphorylation [36]. This causes arterial vasodilation which results in increased blood flow due to a decrease in vascular resistance, and a reduction in blood pressure and heart rate. L-theanine's effect on changes in blood pressure under physical and psychological stresses were reported by Yoto et al [37]. In that study, the researchers suggested that L-theanine not only reduced anxiety but also attenuated the rise in blood pressure in high-stress-response adults. L-theanine had no effect on lowering the rise in blood pressure caused by strong physical stress [37]. In a similar study, Kimura et al showed that L-theanine's effect on psychological and physiological stress responses showed that L-theanine intake resulted in a reduction in the heart rate and salivary immunoglobulin A (s-IgA) responses to an acute stress task relative to the placebo controlled condition. The researchers concluded that the reduction in heart rate and s-IgA were likely attributable to an attenuation of sympathetic nervous activation. The researcher suggested that the oral intake of L-theanine could cause anti-stress effects via the inhibition of cortical neuron excitation [38]. L-Theanine promotes alpha wave generation in the brain, resulting in an awake, alert, relaxed physical and mental condition [39,40]. Animal neurochemistry studies suggest that L-theanine increases brain serotonin, dopamine, GABA (gamma-aminobutyric acid) levels and has micromolar affinities for AMPA (α-amino-3-hydroxy-5-methyl-4-isoxazolepropionic acid), Kainate and NMDA (N-methyl-D-Aspartate) receptors [41]. GABA is the most widespread inhibitory neurotransmitter of the brain. Elevated levels of GABA are associated with a relaxed mental state, whereas reduced levels are associated with anxiety. When GABA levels are decreased, there is an augmentation of nerve impulses in the neuron. Theanine which crosses the blood-brain barrier via the large neutral amino acid (leucine-preferring) transport system [42], increases GABA levels in the brain, opposing excess stimulation of nerve impulses by excitatory neurotransmitters such as glutamate, resulting in a state of well-being and relaxation. L-theanine's calming effect demonstrates theanine's effectiveness in stress management which would be beneficial in cardiac patients. Thrombin is a serine protease in the blood plasma that causes coagulation of blood by converting fibrinogen to fibrin. Theanine is a potent inhibitor of thrombin-stimulated thromboxane formation in whole blood [43], and is responsible for theanine's anticoagulant property. Ali et al showed that theanine inhibited thromboxane formation in rabbit whole blood stimulated by thrombin [43]. Inhibition of thromboxane formation by theanine would be expected to significantly reduce the median platelet aggregation inhibition time.

Anti-apoptotic properties of L-Theanine: L-Theanine's anti-apoptosis properties were demonstrated by Li et al. The researchers showed that L-theanine protects human hepatic L02 cells from hydrogen peroxide-induced apoptosis and suggested that L-theanine could protect the L02 cells against H₂0₂-induced apoptosis via suppression of p38 mitogen-activated protein kinase [44]. The researchers concluded that L-theanine prevented L02 cells from H₂0₂-induced apoptosis through p3 8 signaling pathway which may be related to its inhibitory effect on reactive oxygen species (ROS) [44].

Phenolic acids are naturally occurring compounds found in the plant kingdom [45]. More than 500 plants contain Protocatechuic acid (PCA) [45]. Protocatechuic acid (3,4-dihydroxybenzoic acid) is a phenolic compound and is a major metabolite of antioxidant polyphenols found in green tea and in many food plants such as Olea europaea (olives), Vitis vinifera (white wine grapes) and Hibiscus sabdariffa (roselle) [46,47,48,49]. Roselle is used worldwide in food and beverages [50]. Mushrooms such as Agaricus bisporus [51], and Phellinus linteas [52], contains protocatechuic acid. Acai oil obtained from the fruit of the acai palm (Euterpe oleracea) [48], and carrots (Daucus carota) [45] are rich in protocatechuic acid. Protocatechuic acid also occurs in rich quantity in various fruits such as berries (raspberry, blueberry, mulberry, strawberry, cranberry, gooseberry) and loquat fruits, wine and honey [45].

Protocatechuic aldehyde (3,4-dihydroxybenzaldehyde) is a naturally occurring phenolic aldehyde that is found in barley, green cavendish bananas, grapevine leaves, and root of the herb Salvia Miltiorrhiza [53].

Phenolic compounds (Phenols) consist of one or more hydroxyl groups bounded directly to an aromatic hydrocarbon group. Table 2 shows the six isomeric compounds of dihydroxybenzoic acids which have two-OH groups and one carboxylic group on the benzene ring [54].

TABLE 2
Six isomeric compounds of dihydroxybenzoic acids
2,3-Dihydroxybenzoic acid
(2-Pyrocatechuic acid)

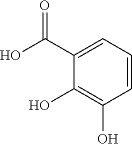 [SMILES: O=C(O)c1cccc(O)c1O]

CAS RN: 303-38-8
2,4-Dihydroxybenzoic acid
(beta-Resorcylic acid)

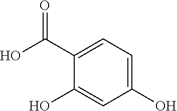 [SMILES: O=C(O)c1ccc(O)cc1O]

CAS RN: 89-86-1
2,5-Dihydroxybenzoic acid
(Gentisic acid)

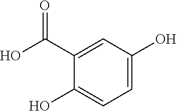 [SMILES: O=C(O)c1cc(O)ccc1O]

CAS RN: 490-79-9
2,6-Dihydroxybenzoic acid
(gamma-Resorcylic acid)

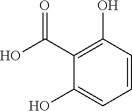 [SMILES: O=C(O)c1c(O)cccc1O]

CAS RN: 303-07-1
3,4-Dihydroxybenzoic acid
(Protocatechuic acid)

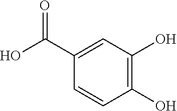 [SMILES: O=C(O)c1ccc(O)c(O)c1]

CAS RN: 99-50-3
3,5-Dihydroxybenzoic acid
(alpha-Resorcylic acid)

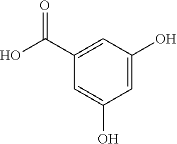 [SMILES: O=C(O)c1cc(O)cc(O)c1]

CAS RN: 99-10-5

Protocatechuic acid is a gray to tan solid crystalline powder with a mild phenolic odor [45]. Protocatechuic acid has a chemical abstract service (CAS) registry number of 99-50-3, molecular formula C₇H₆O₄, molecular weight 154.12 g/mol, pKa of 4.26, melting point 221° C., solubility in water of 18.2 mg/ml, oral LD₅₀ of greater than 3000 mg/kg in rabbits [55] and FEMA GRAS Number 4430.
Scheme 1 illustrates protocatechuic acid biosynthesis [56]. Biosynthesis enzymes include 3-dehydroshikimate dehydratase, (3S,4R)-3,4-dihydroxycyclohexa-1,5-diene-1,4-dicarboxylate dehydrogenase, terephthalate 1,2-cis-dihydrodiol dehydrogenase, 3 -hydroxybenzoate 4-monooxygenase, 4-hydroxybenzoate 3 -monooxygenase (NAD(P)H), 4-sulfobenzoate 3,4-dioxygenase, vanillate monooxygenase, 3,4-dihydroxyphthalate decarboxylase, and 4,5-dihydroxyphthalate decarboxylase. Degradation enzymes include the enzyme protocatechuate decarboxylase, which uses 3,4-dihydroxybenzoate to produce catechol and CO₂, and the enzyme protocatechuate 3,4-dioxygenase, which uses 3,4-dihydroxybenzoate and O₂ to produce 3-carboxy-cis, cis-muconate.

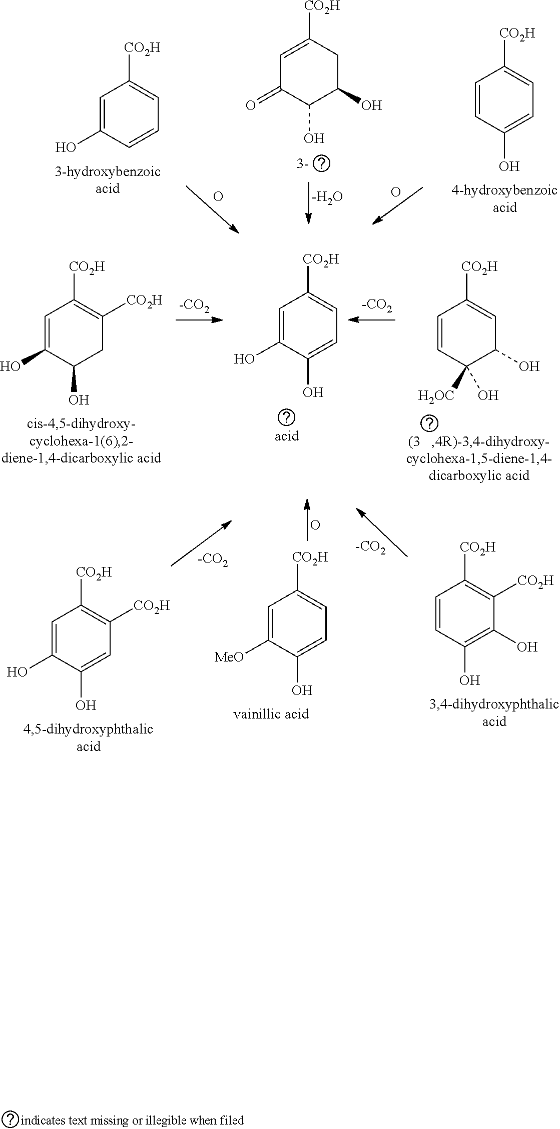

Antioxidant properties of Protocatechuic acid: Protocatechuic acid was shown to have free radical scavenging and antioxidant activities by decreasing lipid peroxidation and increasing the scavenging of hydrogen peroxide (H₂O₂) and diphenylpicrylhydrazl (DPPH) [46,57]. In J77A.1 macrophage, protocatechuic acid decreased oxidized low-density lipoprotein levels (LDL), inhibited superoxide (O₂¹) and H₂O₂ production, and also restored glutathione (GSH) related enzymes via c-Jun N-terminal kinase (JNK) mediated nuclear factor (erythroid-derived 2) like 2 (Nrf2) activation [46,58,59]. Protocatechuic acid also reduced reactive oxygen species (ROS) induced apoptosis by improving mitochondrial function, inhibiting DNA fragmentation in H₂O₂-induced oxidative stress in human neuronal cells [46,60], preventing lactate dehydrogenase (LDH) release in H₂O₂-induced oxidative stress in PC12 cells [46,61], and inhibiting intracellular ROS level in BNLCL2 cells [46,62]. In streptozotocin (STZ) induced diabetic rats, protocatechuic acid was found to decrease ROS formation in liver, heart, kidney, and brain by restoring endogenous antioxidant enzyme activities [46,49,63].

Antiatherosclerotic and Antihyperlipidemic properties of Protocatechuic Acid: Protocatechuic acid has been found to possess the antiatherosclerotic effect. Protocatechuic acid inhibits monocyte adhesion to tumor necrosis factor-α (TNF-α)-activated mouse aortic endothelial cells, which is associated with the inhibition of vascular cell adhesion molecule 1 (VCAM-1) and intercellular adhesion molecule 1 (ICAM-1) expression and reduces NF-κB binding activity. Protocatechuic acid possesses the antiatherogenic effect by virtue of its anti-inflammatory activity [45,64].

Anti-inflammatory properties of Protocatechuic acid: Protocatechuic acid was shown to suppress tumor necrosis factor alpha (TNF-α), interleukin 1 beta (IL-1β), inducible nitric oxide synthase (iNOS), and cyclooxygenase 2 (COX-2) expression via the regulation of the nuclear factor kappa-light-chain-enhancer of activated B cells (NF-kB) and mitogen-activated protein kinase (MAPK) activation in lipopolysaccharide-(LPS-) induced RAW 264.7 cell damage [46,65]. In vitro reports, animal studies demonstrated that protocatechuic acid strongly inhibited inflammation by inhibiting carrageenan-induced inflammation in mice by decreasing TNF-α, IL-1β, and prostaglandin E2 (PGE₂) levels, suppressed iNOS and COX-2 expression in apolipoprotein E (ApoE) deficient mice [46,65], prevented LPS-induced sepsis in mice via decreased NO levels and suppressed IL-10 [46,66], reduced VCAM-1 and ICAM-1 [46,67]. Protocatechuic acid treatments at 2% and 4% in diabetic mice was shown to reduce interleukin-6, tumor necrosis factor-α, and monocyte chemoattractant protein-1 levels in the heart and kidney [68]. Inflammation plays an important role in cardiovascular disease [69]. Cesari et al examined the predictive value of the inflammatory marker interleukin-6 (IL-6) for the incidence of CHD in subjects 70 to 79 years old without evidence of cardiovascular disease at baseline. In their study, compared with traditional risk factors, increased IL-6 level was the strongest and most consistent risk factor for cardiovascular events [69]. Protocatechuic acid sulfates have been shown to reduce the production of pro-inflammatory biomarkers of coronary risk, such as interleukin-6 (IL-6) in human endothelial cells [70,71].

Anticoagulant properties of Protocatechuic acid: Lin, et al showed that protocatechuic acid treatments at 2% and 4% in diabetic mice significantly lowered plasminogen activator inhibitor-1 activity and fibrinogen level; increased plasma activity of antithrombin-III and protein C; and decreased triglyceride content in plasma, heart, and liver [45,68].

Antihyperglycemic properties of protocatechuic acid: It has been demonstrated that peroxisome proliferator-activated receptor gamma (PPARγ) is one of several targets of insulin activity, which regulates the expression and activity of key players in the maintenance of glucose transport machinery efficiency, such as glucose transporter (GLUT) 4 and adiponectin [46,72,73]. In in vitro studies, protocatechuic acid has been shown to exert an insulin-like activity in oxidized LDL-induced insulin resistance in adipocytes via increased PPARγ activation [46,73]. Similarly, in vivo studies also demonstrated that protocatechuic acid decreased blood glucose levels in STZ-induced diabetes via restored carbohydrate metabolic enzyme activity, increased plasma insulin level, and normalized the activity of pancreatic islets [46,49,63,74]. Bhattacharjee et al showed that protocatechuic acid suppresses diabetic cardiomyopathy signaling molecules, PI3K, IRS, Akt, AMPK PKC, NF-kB and PARP, involved in glucose utilization and inflammatory pathophysiology [75]. Lin et al showed that protocatechuic acid at 1%, 2%, and 4% when given to diabetic mice for 8 weeks, significantly lowered plasma glucose and increased insulin levels [68].

Anti-apoptotic and Pro-apoptotic properties of protocatechuic acid: Polyphenols have been shown to improve cell survival and protect against cytotoxicity by inhibiting apoptosis [46,76]. However, they can also induce apoptosis and prevent tumor growth [46,77,78]. These opposite effects are mainly due to its effects on the controlling of the cell redox state [46]. Evidence from in vitro studies revealed that PCA has cell-protective effects via increased IkB degradation and subsequent NF-kB activation in TNF-α-induced cell death [46,79], attenuated changes of the mitochondrial membrane permeability, decreased oxidative stress damage and increased Bcl-2 levels in 1-methyl-4-phenylpyridinium-(MPP+-) induced apoptotic cell death [46,80], decreased caspase-3 activity in isolated neuronal stem cells (NSCs) [46,81], and reduced LDH leakage in H₂O₂-induced apoptosis [46,82]. In MPP+-induced cell death, PCA treatment resulted in a return to normal cellular morphology and normal mitochondria [46,83]. Moreover, PCA has been shown to have cell-protective effects via antioxidant and scavenging activities [46,84].

Antifibrotic properties of Protocatechuic aldehyde: Studies have shown protocatechuic aldehyde to possess beneficial antifibrogenic effects. Transforming growth factor-β1 (TGF-β1) and connective transforming growth factor (CTGF) are associated with the pathophysiology of liver fibrosis [45,85]. In carbon tetrachloride (CCL₄) induced rat liver fibrosis model, liver fibrosis grade, and histopathological changes were evaluated, and biochemical indicators were determined [45,85]. Protocatechuic aldehyde was seen to inhibit the levels of TGF-β1, CTGF inhibit HSCs proliferation, type I collagen, and type III collagen in TNF-α stimulated HSCs [45,85] also, it causes significant reduction in fibrosis grade, ameliorates biochemical indicators, and histopathological morphology and reduces liver TGF-β1 and CTGF expression [45,85].

TABLE 3
Protocatechuic Aldehyde
3,4-dihydroxy benzaldehyde
(Protocatechuic aldehyde)

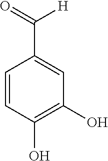

CAS RN: 139-85-5

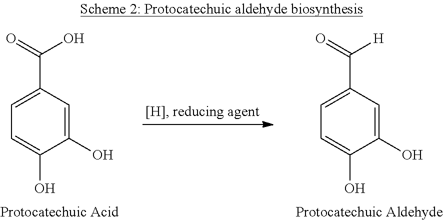 [SMILES: O=C(O)c1ccc(O)c(O)c1 ; O=Cc1ccc(O)c(O)c1]

Neuroprotective properties of L-Theanine and Protocatechuic Acid: Glutamate is the most abundant and widely available excitatory neurotransmitter in the central nervous system [86]. The increase in extracellular glutamate levels may induce a massive calcium ion influx and enhance formation of ROS (Reactive Oxygen Species), eventually leading to neuronal cell death [86,87]. Nozawa et al. found that 50% of neurons died in vitro when exposed to glutamate in a certain concentration [86,88]. However, if pretreated with theanine, the probability of death was significantly decreased. Recent evidence suggests that the glutamate-glutamine cycle formed between neurons and astrocytes is an important pathway for the regulation of glutamate concentration in the brain [86,89]. Theanine can inhibit the transport of glutamine and regulate the glutamate-glutamine cycle in the neurons [86,90] and, thus, provide neuroprotection. In addition, theanine could promote neurogenesis by increasing the expression of glutamine transporter slc38a1[86,91,92]. The results further suggest that theanine may regulate the extracellular glutamine concentration in the neurons and by doing so may exert neuroprotective effects on neurodegenerative diseases. Excessive ROS generation or impairment in body antioxidant defense system induce oxidative stress which is known to be related with the pathogenesis of human neurodegenerative diseases [93,94,95]. It is due to the fact that the brain is particularly sensitive to the effects of ROS because has a high oxygen demand, relatively low antioxidant activity and contains a lot of lipid cells, which are susceptible to peroxidation. All these factors make neurons and glia highly susceptible to destruction by reactive oxygen or nitrogen species and neurodegeneration [93,95,96]. Protocatechuic acid easily crosses the blood brain barrier [93]. It is believed that PCA exhibits neuroprotective effects through an improvement of the endogenous antioxidant enzymatic system [93,97]. The free radical scavenging properties of PCA were also analyzed in aqueous and lipid solutions by Galano and Pérez-González. PCA was evaluated as moderately good anti-radical protector in lipid solutions (non-polar environment) and as an excellent peroxyl radical scavenger in the aqueous solutions (polar environment) [93,98]. Another advantageous feature of PCA in neurodegenerative diseases may be its anti-apoptotic activity [93]. In chronic intermittent hypoxic rats PCA was found to restore expression of the BDNF (Brain-Derived Neurotrophic Factor) and increase its level in both hippocampus and the prefrontal cortex of rats, thereby supporting reduction of the neuronal apoptosis and an increased synaptic plasticity [93,99]. PCA's role as an anti-inflammatory in neurodegenerative diseases includes the inhibitory effects of PCA on the protein expression of pro-inflammatory cytokines: tumor necrosis factor α (TNF-α) and interleukin-1β (IL-1β) [65], down-regulating the inflammatory enzymes, COX-2 and iNOS [65]. Inhibition of the NF-κB activation and its translocation to the nucleus. Moreover, PCA counteracted the phosphorylation of ERK-1, ERK-2, JNK-1, JNK-2 and p38 MAPKs in LPS-stimulated RAW 264.7 cells and was the one, which most potently inhibited those inflammatory mediators both in vivo and in vitro among all agents analyzed [65,93] and inhibition of MAPK activation [93,100]. The role of Reactive Oxygen Species, pro-inflammatory cytokines, and antioxidants in regulating the levels of microRNAs in Parkinson's Disease was evaluated by Prasad [101]. In that study, Prasad demonstrated that ROS and pro-inflammatory cytokines cause neurodegeneration by altering the levels of microRNAs, and that antioxidants can protect neurons by reducing oxidative stress and chronic inflammation by altering the levels of microRNAs [101]. As such, cocrystal antioxidants of PCA with L-Theanine may play a significant role in the treatment of neurodegenerative diseases.

The various features of novelty which characterize the invention are pointed out with particularity in the claims annexed to and forming a part of this disclosure. For a better understanding of the invention, its operation, advantages, and specific objects attained by its uses, reference is made to the descriptive matter in which a preferred embodiment of the invention is illustrated.

Accordingly, it is an object of the invention to provide a composition comprising cocrystals of natural protocatechuic acid with L-theanine.

In one embodiment, the cocrystals of the invention are natural antioxidants. In one embodiment, the cocrystals of the invention are natural anti-inflammatory agents. In one embodiment, the cocrystals of the invention are water soluble.

Another object of the invention is to provide a method of manufacturing comprising a cocrystal composition of protocatechuic acid with L-theanine. In one embodiment, the method comprises grinding a mixture containing protocatechuic acid and L-theanine and an alcohol solution in an agate mortar. In one embodiment, the alcohol solution is 70% isopropanol. In one embodiment, the grinding is repeated until the mixture is dry. In some embodiments, the mixture forms a slurry after grinding.

Embodiments of the invention may include natural water soluble cocrystal compositions of protocatechuic acid and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R1-group [C₄ or gamma-CH₂—C(O)—NR₁] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In some embodiments, the invention relates to a cocrystal protocatechuic acid and L-theanine formulation. In some embodiments, the formulation is a sublingual formulation. In some embodiments, the formulation can bypass the hepatic first pass effect, with enhanced dissolution rate and bioavailability when a rapid onset of action is desired. In some embodiments, the formulation is water soluble and crosses the blood-brain barrier. In some embodiments, the formulation has anti-angiogenic properties. In some embodiments, the formulation has anticoagulant properties. In some embodiments, the formulation has anti-apoptotic properties. In some embodiments, the formulation has anti-hyperglycemic properties. In some embodiments, the formulation has anti-hyperlipidemic properties. In some embodiments, the formulation has anti-atherosclerotic properties. In some embodiments, the formulation has cardioprotective properties. In some embodiments, the formulation has neuroprotective properties.

Another object of the invention is to provide a cocrystal protocatechuic aldehyde and L-theanine formulation. In one embodiment, the formulation has anti-fibrotic properties.

Another object of the invention is to provide a composition that includes at least one monohydroxybenzoic acid or dihydroxybenzoic acid, and L-theanine. Non-limiting examples of a monohydroxybenzoic acid include 2-hydroxybenzoic acid, 3-hydroxybenzoic acid, or 4-hydroxybenzoic acid. Non-limiting examples of a dihydroxybenzoic acid include 2,3-dihydroxybenzoic acid, 2,4-dihydroxybenzoic acid, 2,5-dihydroxybenzoic acid, 2,6-dihydroxybenzoic acid, 3,4-dihydroxybenzoic acid, and 3,5-dihydroxybenzoic acid.

In some embodiments, the theanine enantiomer may be L-theanine, D-theanine, or D,L-theanine.

In some embodiments, the theanine enantiomer may be an alpha variant of theanine or a beta variant of theanine.

In some embodiments, the alpha variant of theanine may be L-northeanine, D-northeanine, DL-northeanine, L-homotheanine, D-homotheanine, DL-homotheanine, L-bishomotheanine, D-bishomotheanine, or D,L-bishomotheanine.

In some embodiments, the alpha variant of theanine is a homologous analog of theanine.

In some embodiments, the alpha variant of theanine contains a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, the theanine enantiomer is a racemic mixture of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments the theanine enantiomer is an S enantiomer of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, the theanine enantiomer is an R enantiomer of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, in addition to L-theanine, other analogues include D-theanine, racemic theanine or D, L-theanine and its congeners including beta and reverse beta amino acid forms, shortened or nor-theanine (aspartic acid analogue), and the lengthened homo-theanines and their isomers. Further, gamma alkylamido analogues extend a full range of molecular property for drug cocrystals.

In one embodiment, the disclosure relates to a composition including protocatechuic acid and L-theanine. In one embodiment, the theanine is L-theanine. In some embodiments, the wt. % of L-theanine in the composition is between about 50% and about 60%. In some embodiments, the wt. % of L-theanine in the composition is selected from the group consisting of about 50.00%, about 50.10%, about 50.20%, about 50.30%, about 50.40%, about 50.50%, about 50.60%, about 50.70%, about 50.80%, about 50.90%, about 51.00%, about 51.10%, about 51.20%, about 51.30%, about 51.40%, about 51.50%, about 51.60%, about 51.70%, about 51.80%, about 51.90%, about 52.00%, about 52.10%, about 52.20%, about 52.30%, about 52.40%, about 52.50%, about 52.60%, about 52.70%, about 52.80%, about 52.90%, about 53.00%, about 53.10%, about 53.20%, about 53.30%, about 53.40%, about 53.50%, about 53.60%, about 53.70%, about 53.80%, about 53.90%, about 54.00%, about 54.10%, about 54.20%, about 54.30%, about 54.40%, about 54.50%, about 54.60%, about 54.70%, about 54.80%, about 54.90%, about 55.00%, about 55.10%, about 55.20%, about 55.30%, about 55.40%, about 55.50%, about 55.60%, about 55.70%, about 55.80%, about 55.90%, about 56.00%, about 56.10%, about 56.20%, about 56.30%, about 56.40%, about 56.50%, about 56.60%, about 56.70%, about 56.80%, about 56.90%, about 57.00%, about 57.10%, about 57.20%, about 57.30%, about 57.40%, about 57.50%, about 57.60%, about 57.70%, about 57.80%, about 57.90%, about 58.00%, about 58.10%, about 58.20%, about 58.30%, about 58.40%, about 58.50%, about 58.60%, about 58.70%, about 58.80%, about 58.90%, about 59.00%, about 59.10%, about 59.20%, about 59.30%, about 59.40%, about 59.50%, about 59.60%, about 59.70%, about 59.80%, about 59.90%, and about 60.00%.

In some embodiments, the wt. % of protocatechuic acid in the composition is between about 40% and about 50%. In some embodiments, the wt. % of L-theanine in the composition is selected from the group consisting of about 40.00%, about 40.10%, about 40.20%, about 40.30%, about 40.40%, about 40.50%, about 40.60%, about 40.70%, about 40.80%, about 40.90%, about 41.00%, about 41.10%, about 41.20%, about 41.30%, about 41.40%, about 41.50%, about 41.60%, about 41.70%, about 41.80%, about 41.90%, about 42.00%, about 42.10%, about 42.20%, about 42.30%, about 42.40%, about 42.50%, about 42.60%, about 42.70%, about 42.80%, about 42.90%, about 43.00%, about 43.10%, about 43.20%, about 43.30%, about 43.40%, about 43.50%, about 43.60%, about 43.70%, about 43.80%, about 43.90%, about 44.00%, about 44.10%, about 44.20%, about 44.30%, about 44.40%, about 44.50%, about 44.60%, about 44.70%, about 44.80%, about 44.90%, about 45.00%, about 45.10%, about 45.20%, about 45.30%, about 45.40%, about 45.50%, about 45.60%, about 45.70%, about 45.80%, about 45.90%, about 46.00%, about 46.10%, about 46.20%, about 46.30%, about 46.40%, about 46.50%, about 46.60%, about 46.70%, about 46.80%, about 46.90%, about 47.00%, about 47.10%, about 47.20%, about 47.30%, about 47.40%, about 47.50%, about 47.60%, about 47.70%, about 47.80%, about 47.90%, about 48.00%, about 48.10%, about 48.20%, about 48.30%, about 48.40%, about 48.50%, about 48.60%, about 48.70%, about 48.80%, about 48.90%, about 49.00%, about 49.10%, about 49.20%, about 49.30%, about 49.40%, about 49.50%, about 49.60%, about 49.70%, about 49.80%, about 49.90%, and about 50.00%.

In some embodiments, the composition comprises one or more of a binder, an emulsifier, and a disintegrant.

In some embodiments, the composition further includes a sugar alcohol.

In some embodiments, the sugar alcohol has a configuration of the L-configuration or the D-configuration.

In some embodiments, the theanine enantiomer further includes a carbohydrate functional group thereon. In some embodiments, the carbohydrate functional group may be of the L-configuration or the D-configuration. In these embodiments, the carbohydrates employed may be monosaccharides, disaccharides, trisaccharides, oligosaccharides or polysaccharides.

In some embodiments, the theanine enantiomer further comprises an amino acid functional group thereon. In some embodiments, the amino acid functional group is a dipeptide.

In one embodiment, the disclosure relates to a dosage form including protocatechuic acid and L-theanine. In some embodiments, the amount of protocatechuic acid in the dosage form is between about 300 mg and about 450 mg. In some embodiments, the amount of protocatechuic acid in the dosage form is between about 25 mg and about 600 mg. In some embodiments, the amount of protocatechuic acid in the dosage form is between about 100 mg and about 400 mg. In some embodiments, the amount of protocatechuic acid in the dosage form is selected from the group consisting of about 200 mg, about 205 mg, about 210 mg, about 215 mg, about 220 mg, about225 mg, about230 mg, about235 mg, about240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 376 mg, about 379 mg, about 380 mg, about 381 mg, about 395 mg, about 390 mg, about 395 mg, about 400 mg, about 405 mg, about 410 mg, about 415 mg, about420 mg, about 425 mg, about 430 mg, about 435 mg, about 440 mg, about 445 mg, about 450 mg, about 301 mg, about 306 mg, and about 441 mg.

In some embodiments, the amount of L-theanine in the dosage form is between about 300 mg and about 550 mg. In some embodiments, the amount of L-theanine in the dosage form is between about 200 mg and about 300 mg. In some embodiments, the amount of L-theanine in the dosage form is selected from the group consisting of about 200 mg, about 205 mg, about 210 mg about 215 mg, about 220 mg, about225 mg, about230 mg, about235 mg, about240 mg, about 245 mg, about 250 mg, about 255 mg, about 260 mg, about 265 mg, about 270 mg, about 275 mg, about 280 mg, about 285 mg, about 290 mg, about 295 mg, 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 376 mg, about 379 mg, about 380 mg, about 381 mg, about 395 mg, about 390 mg, about 395 mg, about 400 mg, about 405 mg, about 410 mg, about 415 mg, about420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 342 mg, about 347 mg, and about 504 mg.

In some embodiments, the dosage form comprises a composition comprising protocatechuic acid and L-theanine, wherein protocatechuic acid and L-theanine are in the form of a cocrystal. In some embodiments, the dosage form can be blended with a vitamin water drink with electrolytes or any type of fruit drink.

In some embodiments, the invention relates to one or more of the following cocrystal products:

| | |
|---|---|
| Cocrystal#1 | |
| Protocatechuic Acid | 441 mg (46.4%) |
| Theanine | 504 mg (53.6%) |
| Cocrystal#2 | |
| Protocatechuic Acid | 301 mg (46.8%) |
| Theanine | 342 mg (53.2%) |
| Cocrystal#3 | |
| Protocatechuic Acid | 306 mg (46.9%) |
| Theanine | 347 mg (53.1%) |

A further object of the invention is to provide method of treating a disease or disorder in a subject in need thereof. In one embodiment, the method comprises administering to the subject a composition of the invention. In one embodiment, the method comprises administering to the subject a dosage form of the invention.

A further object of the invention is to provide method of administering a water-soluble protocatechuic acid and theanine cocrystal formulation sublingually in humans that provides enhanced dissolution and bioavailability and is suitable for treatment of diseases and disorders.

In a non-limiting example, the disease or disorder is at least one of an acute inflammatory disease, a chronic inflammatory disease, a cardiovascular inflammatory disease, oxidative stress, diabetes, diabetic retinopathy, a hypercoagulable disorder, apoptosis, and a neurodegenerative disease. In one embodiment, the hypercoagulable disorder is associated with thrombin-stimulated thromboxane formation

In one embodiment, the invention relates to a natural water-soluble cocrystal formulation of protocatechuic acid and L-theanine for the treatment of diseases or disorders associated with acute inflammation.

In one embodiment, the invention relates to a natural water-soluble cocrystal formulation of protocatechuic acid and L-theanine for the treatment of diseases or disorders associated with chronic inflammation.

In one embodiment, the invention relates to a natural water-soluble cocrystal formulation of protocatechuic acid and L-theanine for the treatment of diseases or disorders associated with cardiovascular inflammatory disease.

In one embodiment, the invention relates to a natural water-soluble cocrystal formulation of protocatechuic acid and L-theanine useful as an antioxidant for the treatment of oxidative stress.

In one embodiment, the invention relates to a natural water-soluble cocrystal formulation of protocatechuic acid and L-theanine for the treatment of a hypercoagulable disorder associated with thrombin-stimulated thromboxane formation, for which the treatment is expected to significantly reduce median platelet aggregation.

In one embodiment, the invention relates to a natural water-soluble cocrystal formulation of protocatechuic acid and L-theanine for the treatment of diseases or disorders associated with atherosclerosis.

In one embodiment, the invention relates to a natural water-soluble cocrystal formulation of protocatechuic acid and L-theanine for the treatment of diabetes and its complication of diabetic retinopathy.

In one embodiment, the invention relates to a natural water-soluble formulation of protocatechuic aldehyde and L-theanine for the treatment of diseases or disorders associated with fibrosis.

In one embodiment, the invention relates to a natural water-soluble cocrystal formulation of protocatechuic acid and L-theanine for the treatment of apoptosis.

In one embodiment, the invention relates to a natural water-soluble cocrystal formulation of protocatechuic acid and L-theanine for the treatment of a neurodegenerative disease.

In some embodiments, a composition of the invention is administered in multiple doses for treating a disease or disorder. In an embodiment, a composition of the invention is administered in multiple doses. In some embodiments, a composition of the invention is administered in multiple doses by oral administration. In some embodiments, a composition of the invention is administered in multiple doses via the sublingual route. In some embodiments, a composition of the invention is administered in multiple doses via intravenous infusion. In some embodiments, dosing is once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In some embodiments, a composition of the invention is administered about once per day to about six times per day. In some embodiments a composition of the invention is administered once daily, while in other embodiments a composition of the invention is administered twice daily, and in other embodiments a composition of the invention is administered three times daily. In some embodiments a composition of the invention is administered three times a week, including every Monday, Wednesday, and Friday. In some embodiments, a composition of the invention is administered at least once a day. In some embodiments, a composition of the invention is administered once a day.

Administration of a composition of the invention may continue as long as necessary. In some embodiments, a composition of the invention is administered for more than 1, 2, 3, 4, 5, 6, 7, 8, 9, 10, 11, 12, 13, 14, 15, 16, 17, 18, 19, 20, 21, 22, 23, 24, 25, 26, 27,28, 29, 30, 31 or more days. In some embodiments, a composition of the invention is administered for less than 28, 14, 7, 6, 5, 4, 3, 2, or 1 day. In some embodiments, a composition of the invention is administered for about 14 days, about 21 days, about 28 days, about 35 days, about 42 days, about 49 days, or about 56 days. In some embodiments, a composition of the invention is administered chronically on an ongoing basis—e.g., for the treatment of chronic effects. In another embodiment the administration of a composition of the invention continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, a composition comprising an amount of protocatechuic acid in a dosage form between 25 mg and about 600 mg and/or an amount of L-theanine in a dosage form between about 300 mg and about 550 mg is administered once a day. In some embodiments, a composition comprising an amount of protocatechuic acid in a dosage form between 25 mg and about 600 mg and/or an amount of L-theanine in a dosage form between about 200 mg and about 300 mg is administered once a day. In some embodiments, a composition comprising an amount of protocatechuic acid in a dosage form between about 100 mg and about 400 mg and/or an amount of L-theanine in a dosage form between about 300 mg and about 550 mg L-theanine is administered once a day. In some embodiments, a composition comprising an amount of protocatechuic acid in a dosage form between about 100 mg and about 400 mg and/or an amount of L-theanine in a dosage form between about 200 mg and about 300 mg L-theanine is administered once a day.

In one embodiment, both protocatechuic acid and L-theanine cross the blood brain barrier.

In one embodiment, the compositions of the disclosure can be blended with a vitamin water drink with electrolytes or any type of fruit juice drink.

In some embodiment, derivatives prepared using protocatechuic acid with L-theanine cocrystal composition be administered via the sublingual route, subconjunctival route in the form of eye drops, and orally.

In some embodiments, the pharmaceutical compositions according to embodiments of the present invention may be prepared as oral solids (tablets, oral disintegrating tablets, effervescent tablets, capsules), oral liquids, hard or soft gelatin capsules, microgels, microspheres, microcapsules, quick dissolve, controlled released, modified released, extended release, slow release, sustained release, syrups, suspensions, granules, wafers (films), pellets, lozenges, powders, chewables or crystals.

Embodiments of the invention include water soluble excipients with optimal disintegration properties.

Cocrystals according to embodiments of the invention may be used to improved one or more physical properties, such as solubility, stability, and dissolution rate, of the active pharmaceutical ingredient of a selected treatment or prevention.

The invention is described in further detail by means of examples, without intending to limit the scope of the invention to these examples alone. While a specific embodiment of the invention has been shown and described in detail to illustrate the application of the principles of the invention, it will be understood that the invention may be embodied otherwise without departing from such principles.

Theanine cocrystals are described in U.S. Pat. Nos. 9,603,937, 9,603,938, 9,603,939, 9,289,438, 9,289,439, 9,289,440, 8,685,948, 8,476,250, 8,304,404, and 8,173,625, which are incorporated herein by reference in their entirety.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

### Preparation of Cocrystals

Experimental Details

X-ray powder diffraction (XRPD) patterns were obtained using a Rigaku MiniFlex powder diffraction system, equipped with a horizontal goniometer operating in the θ/2θ mode. The X-ray source was nickel-filtered Kα emission of copper (1.54184 Å). Samples were packed into the sample holder using a back-fill procedure, and were scanned over the range of 3.5 to 40 degrees 2θ at a scan rate of 0.5 degrees 2 θ/min. The intensity scale for all diffraction patterns was normalized so that the relative intensity of the most intense peak in the pattern equaled 100%.

Measurements of differential scanning calorimetry (DSC) were obtained on a TA Instruments 2910 thermal analysis system. Samples having a mass in the range of 1-2 mg were accurately weighed into an aluminum DSC pan, and then covered with an aluminum lid that was inverted and pressed down so as to tightly contain the powder between the top and bottom aluminum faces of the lid and pan. The samples were then heated at a rate of 10° C./min over the temperature range of 25-200° C.

### Preparation of Example 1

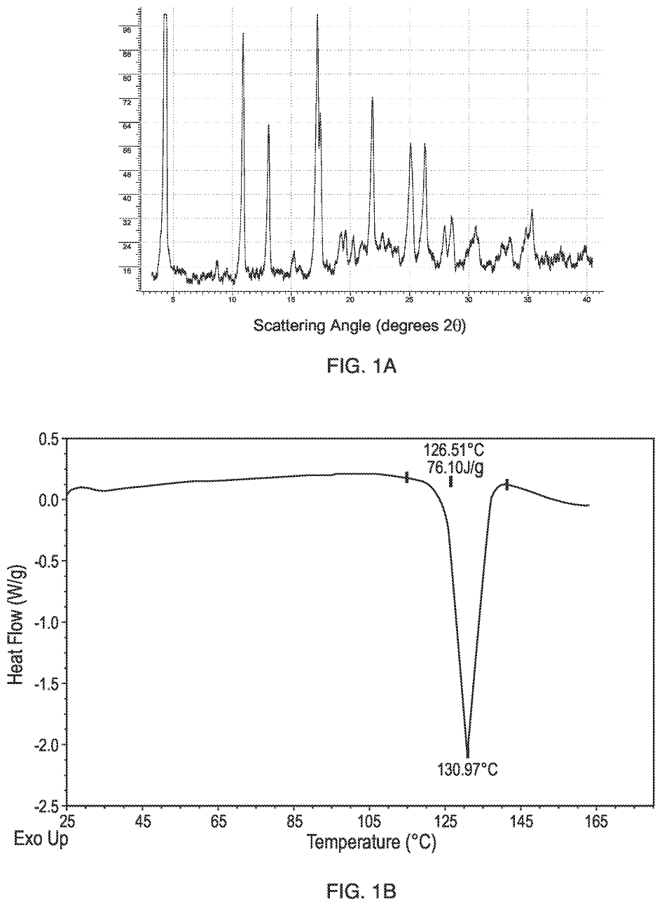

0.313 grams of 2-hydroxybenzoic acid (2.266 mmol) and 0.397 grams of L-theanine (2.279 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 1A, while the DSC thermogram is shown in FIG. 1B. The DSC melting endotherm of the product was characterized by a peak maximum at 131° C. Table 4 shows relevant peaks from the XRDP pattern of the cocrystal product.

TABLE 4
Listing of the 10 Most Intense Scattering Peaks
in the XRPD pattern of the Cocrystal Product Formed
by L-Theanine and 2-Hydroxybenzoic Acid

| Peak | Peak Position | Relative Intensity |
|---|---|---|
| Identification | (degrees 2θ) | (%) |
| 1 | 4.35 | 100.0 |
| 2 | 10.93 | 37.2 |
| 3 | 13.09 | 25.6 |
| 4 | 17.20 | 39.7 |
| 5 | 17.45 | 27.0 |
| 6 | 21.85 | 29.1 |
| 7 | 25.08 | 23.2 |
| 8 | 26.28 | 23.1 |
| 9 | 27.96 | 12.5 |
| 10 | 28.58 | 13.6 |

### Preparation of Example 2

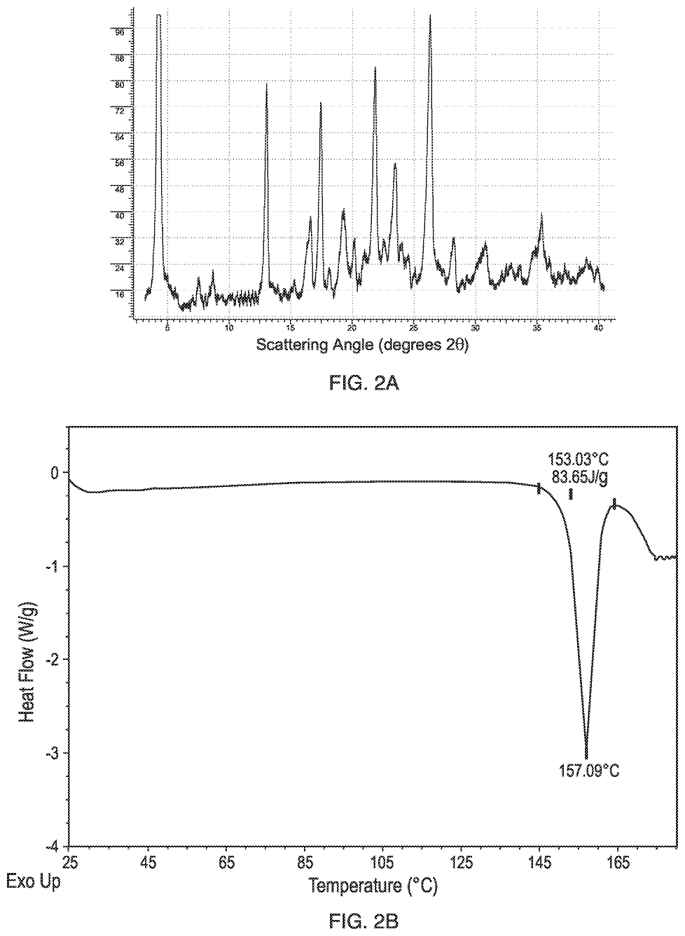

0.304 grams of 3-hydroxybenzoic acid (2.201 mmol) and 0.386 grams of L-theanine (2.216 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 2A, while the DSC thermogram is shown in FIG. 2A. The DSC melting endotherm of the product was characterized by a peak maximum at 157° C. Table 5 shows relevant peaks from the XRDP pattern of the cocrystal product.

TABLE 5
Listing of the 10 Most Intense Scattering Peaks
in the XRPD pattern of the Cocrystal Product Formed
by L-Theanine and 3-Hydroxybenzoic Acid

| Peak | Peak Position | Relative Intensity |
|---|---|---|
| Identification | (degrees 2θ) | (%) |
| 1 | 4.32 | 100.0 |
| 2 | 13.07 | 24.1 |
| 3 | 16.60 | 13.0 |
| 4 | 17.45 | 22.5 |
| 5 | 19.32 | 13.6 |
| 6 | 20.19 | 11.1 |
| 7 | 21.87 | 25.5 |
| 8 | 23.48 | 17.4 |
| 9 | 26.33 | 29.9 |
| 10 | 28.27 | 11.1 |

### Preparation of Example 3

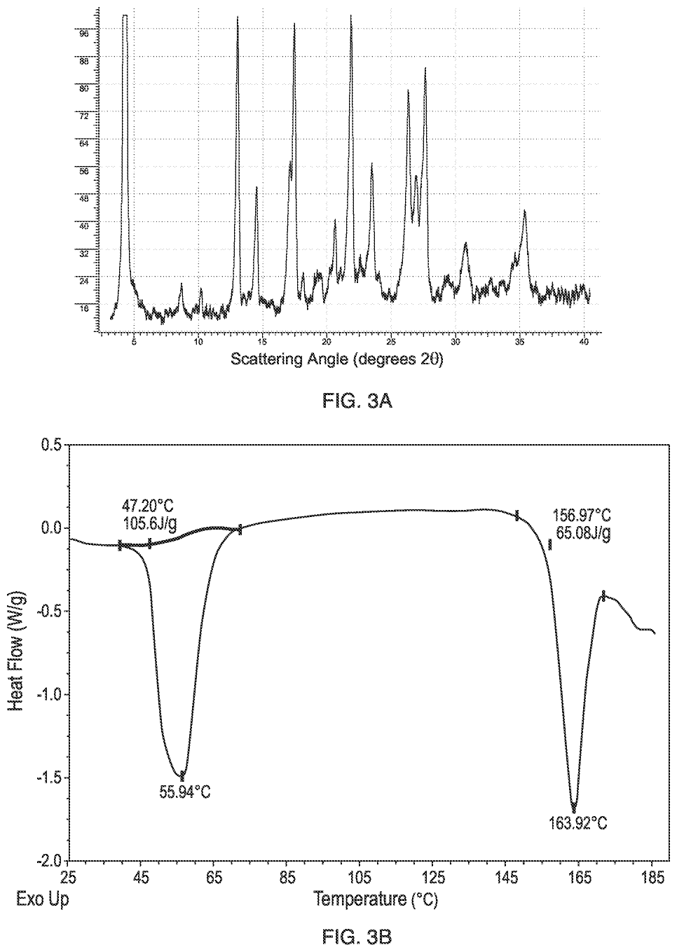

0.309 grams of 4-hydroxybenzoic acid (2.237 mmol) and 0.392 grams of L-theanine (2.250 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 3A, while the DSC thermogram is shown in FIG. 3B. The DSC melting endotherm of the product was characterized by a desolvation endotherm having a peak maximum at 56° C., which was followed by a melting endotherm having a peak maximum at 164° C. Table 6 shows relevant peaks from the XRDP pattern of the cocrystal product.

TABLE 6
Listing of the 10 Most Intense Scattering Peaks
in the XRPD pattern of the Cocrystal Product Formed
by L-Theanine and 4-Hydroxybenzoic Acid

| Peak | Peak Position | Relative Intensity |
|---|---|---|
| Identification | (degrees 2θ) | (%) |
| 1 | 4.30 | 100.0 |
| 2 | 13.06 | 27.5 |
| 3 | 14.54 | 14.8 |
| 4 | 17.48 | 27.0 |
| 5 | 20.63 | 12.4 |
| 6 | 21.88 | 27.6 |
| 7 | 23.50 | 16.8 |
| 8 | 26.33 | 22.0 |
| 9 | 26.93 | 15.9 |
| 10 | 27.66 | 23.8 |

### Preparation of Example 4

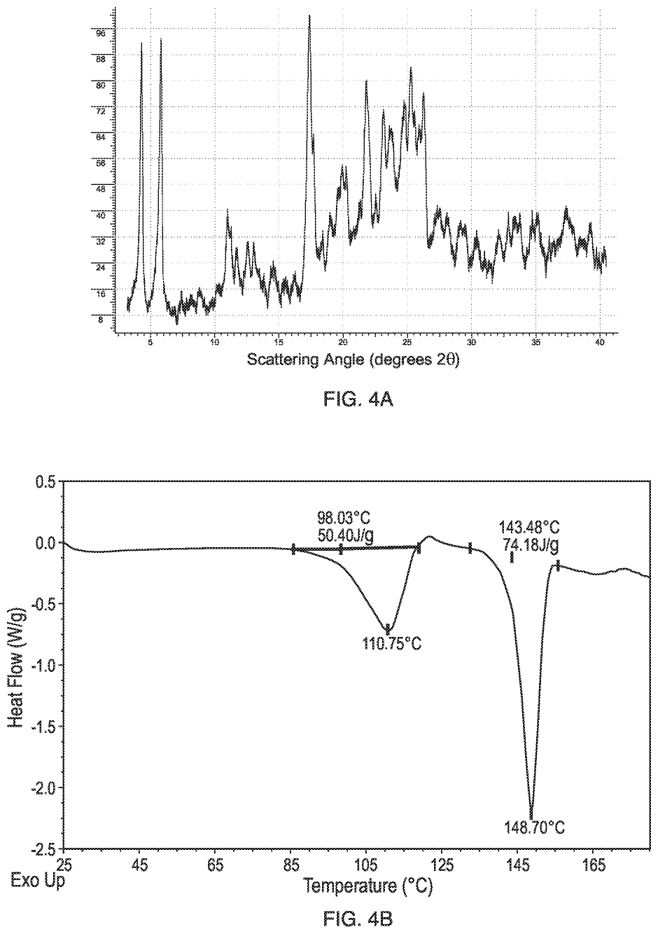

0.320 grams of 2,3-dihydroxybenzoic acid (2.076 mmol) and 0.365 grams of L-theanine (2.095 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 4A, while the DSC thermogram is shown in FIG. 4B. The DSC melting endotherm of the product was characterized by a desolvation endotherm having a peak maximum at 111° C., which was followed by a melting endotherm having a peak maximum at 149° C. Table 7 shows relevant peaks from the XRDP pattern of the cocrystal product.

TABLE 7
Listing of the 10 Most Intense Scattering Peaks
in the XRPD pattern of the Cocrystal Product Formed
by L-Theanine and 2,3-Dihydroxybenzoic Acid

| Peak | Peak Position | Relative Intensity |
|---|---|---|
| Identification | (degrees 2θ) | (%) |
| 1 | 4.30 | 91.6 |
| 2 | 5.81 | 92.8 |
| 3 | 11.01 | 40.5 |
| 4 | 17.37 | 100.0 |
| 5 | 21.81 | 80.2 |
| 6 | 23.15 | 71.0 |
| 7 | 23.70 | 64.8 |
| 8 | 24.80 | 72.4 |
| 9 | 25.29 | 84.0 |
| 10 | 26.26 | 76.4 |

### Preparation of Example 5

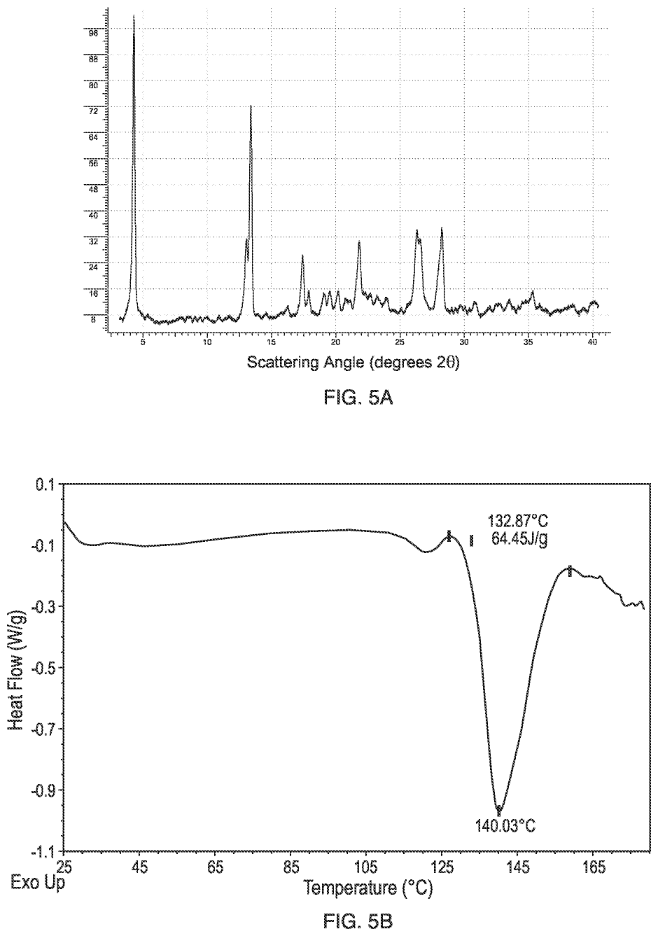

0.313 grams of 2,4-dihydroxybenzoic acid (2.031 mmol) and 0.356 grams of L-theanine (2.044 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 5A, while the DSC thermogram is shown in FIG. 5B. The DSC melting endotherm of the product was characterized by a desolvation endotherm having a peak maximum at 89° C., which was followed by a melting endotherm having a peak maximum at 156° C. Table 8 shows relevant peaks from the XRDP pattern of the cocrystal product.

TABLE 8
Listing of the 10 Most Intense Scattering Peaks
in the XRPD pattern of the Cocrystal Product Formed
by L-Theanine and 2,4-Dihydroxybenzoic Acid

| Peak | Peak Position | Relative Intensity |
|---|---|---|
| Identification | (degrees 2θ) | (%) |
| 1 | 4.29 | 100.0 |
| 2 | 13.39 | 72.3 |
| 3 | 17.41 | 26.4 |
| 4 | 17.90 | 15.4 |
| 5 | 19.07 | 14.8 |
| 6 | 19.52 | 15.1 |
| 7 | 20.17 | 15.2 |
| 8 | 21.83 | 30.7 |
| 9 | 26.32 | 34.2 |
| 10 | 28.25 | 34.8 |

### Preparation of Example 6

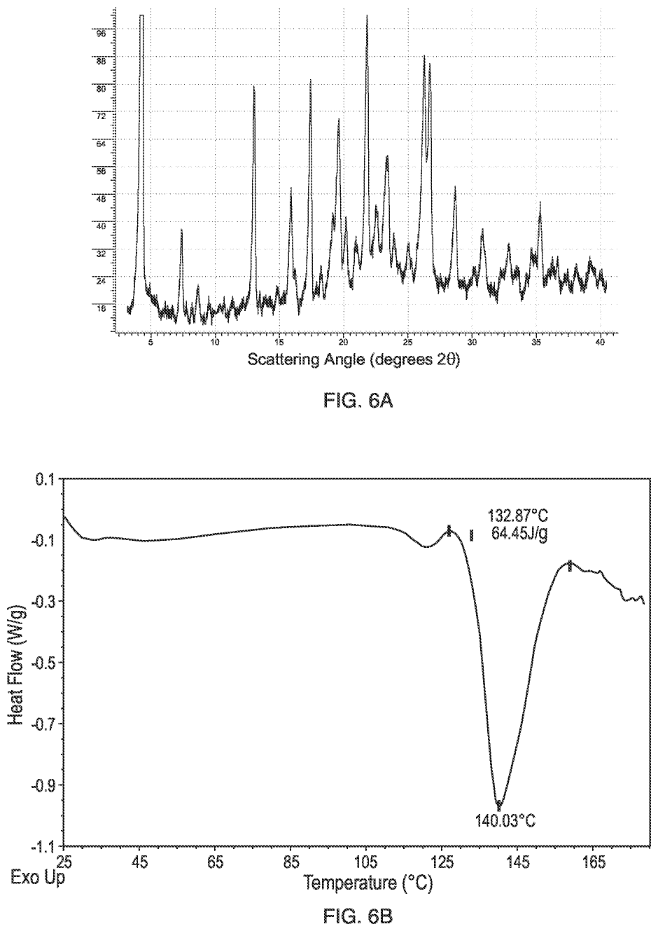

0.322 grams of 2,5-dihydroxybenzoic acid (2.089 mmol) and 0.366 grams of L-theanine (2.101 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 6A, while the DSC thermogram is shown in FIG. 6B. The DSC melting endotherm of the product was characterized by a melting endotherm having a peak maximum at 140° C. Table 9 shows relevant peaks from the XRDP pattern of the cocrystal product.

TABLE 9
Listing of the 10 Most Intense Scattering Peaks
in the XRPD pattern of the Cocrystal Product Formed
by L-Theanine and 2,5-Dihydroxybenzoic Acid

| Peak | Peak Position | Relative Intensity |
|---|---|---|
| Identification | (degrees 2θ) | (%) |
| 1 | 4.30 | 100.0 |
| 2 | 7.34 | 12.0 |
| 3 | 13.04 | 24.4 |
| 4 | 15.89 | 16.3 |
| 5 | 17.42 | 25.1 |
| 6 | 19.62 | 21.9 |
| 7 | 21.82 | 30.4 |
| 8 | 23.35 | 18.6 |
| 9 | 26.30 | 26.6 |
| 10 | 26.70 | 26.4 |

### Preparation of Example 7

0.308 grams of 2,6-dihydroxy-benzoic acid (1.998 mmol) and 0.349 grams of L-theanine (2.003 mmol) were weighed directly into the bowl of an agate mortar, and wetted with 70% isopropanol to form a moderately thick slurry. The slurry was thoroughly ground at the time of mixing, and then periodically re-ground over a period of several days. This cocrystal product was found not to crystallize, and remained in the form of a viscous oil. As a result, it was not possible to measure the XRPD pattern or the DSC thermogram of this product.

### Preparation of Example 8

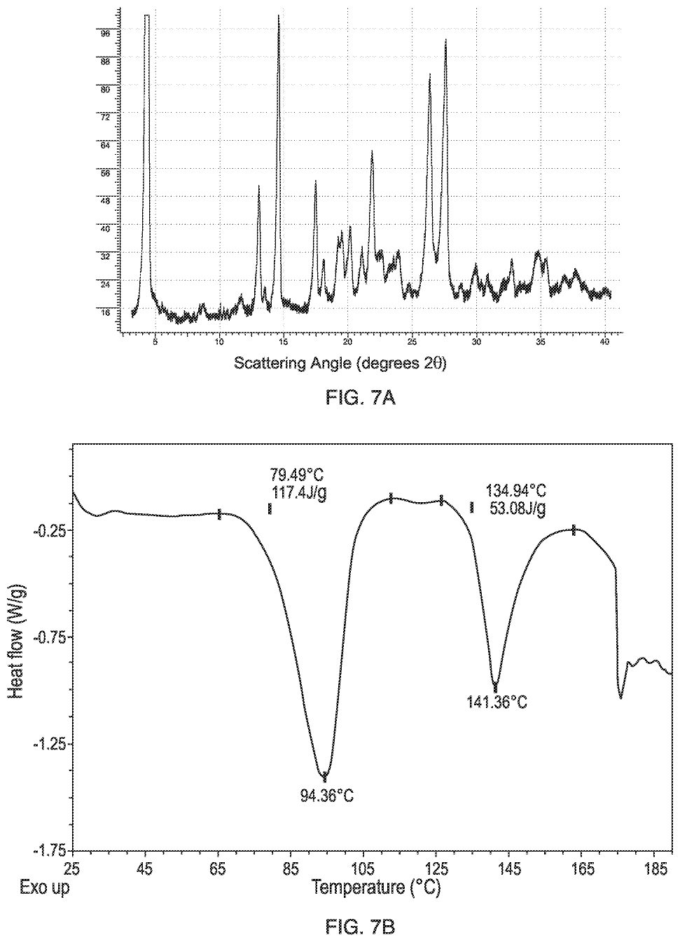

0.301 grams of protocatechuic acid [3,4-dihydroxybenzoic acid] (1.953 mmol) and 0.342 grams of L-theanine (1.963 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 7A, while the DSC thermogram is shown in FIG. 7B. The DSC melting endotherm of the product was characterized by a desolvation endotherm having a peak maximum at 94° C., which was followed by a melting endotherm having a peak maximum at 141° C. Table 10 shows relevant peaks from the XRDP pattern of the cocrystal product.

TABLE 10
Listing of the 10 Most Intense Scattering Peaks
in the XRPD pattern of the Cocrystal Product Formed
by L-Theanine and 3,4-Dihydroxybenzoic Acid
(i.e., Protocatechuic Acid)

| Peak | Peak Position | Relative Intensity |
|---|---|---|
| Identification | (degrees 2θ) | (%) |
| 1 | 4.32 | 100.0 |
| 2 | 13.08 | 29.9 |
| 3 | 14.64 | 61.7 |
| 4 | 17.49 | 34.0 |
| 5 | 19.52 | 25.5 |
| 6 | 20.19 | 29.0 |
| 7 | 21.08 | 22.4 |
| 8 | 21.92 | 45.0 |
| 9 | 26.45 | 75.7 |
| 10 | 27.69 | 86.0 |

### Preparation of Example 9

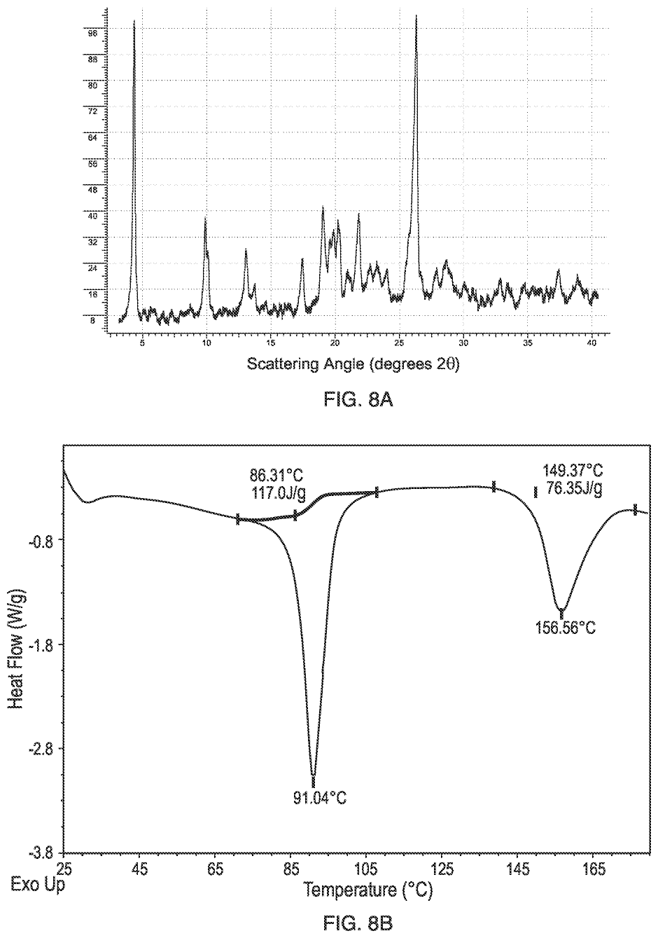

0.306 grams of 3,5-dihydroxybenzoic acid (1.985 mmol) and 0.347 grams of L-theanine (1.992 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the product is shown in FIG. 8A, while the DSC thermogram is shown in FIG. 8B. The DSC melting endotherm of the product was characterized by a desolvation endotherm having a peak maximum at 91° C., which was followed by a melting endotherm having a peak maximum at 157° C. Table 11 shows relevant peaks from the XRDP pattern of the cocrystal product.

TABLE 11
Listing of the 10 Most Intense Scattering Peaks
in the XRPD pattern of the Cocrystal Product Formed
by L-Theanine and 3,5-Dihydroxybenzoic Acid

| Peak | Peak Position | Relative Intensity |
|---|---|---|
| Identification | (degrees 2θ) | (%) |
| 1 | 4.36 | 98.4 |
| 2 | 9.89 | 38.1 |
| 3 | 13.07 | 27.7 |
| 4 | 17.48 | 25.4 |
| 5 | 19.05 | 41.6 |
| 6 | 19.88 | 34.4 |
| 7 | 20.23 | 37.3 |
| 8 | 21.85 | 39.2 |
| 9 | 23.25 | 24.7 |
| 10 | 26.32 | 100.0 |

### Example 2

### Solubility Study

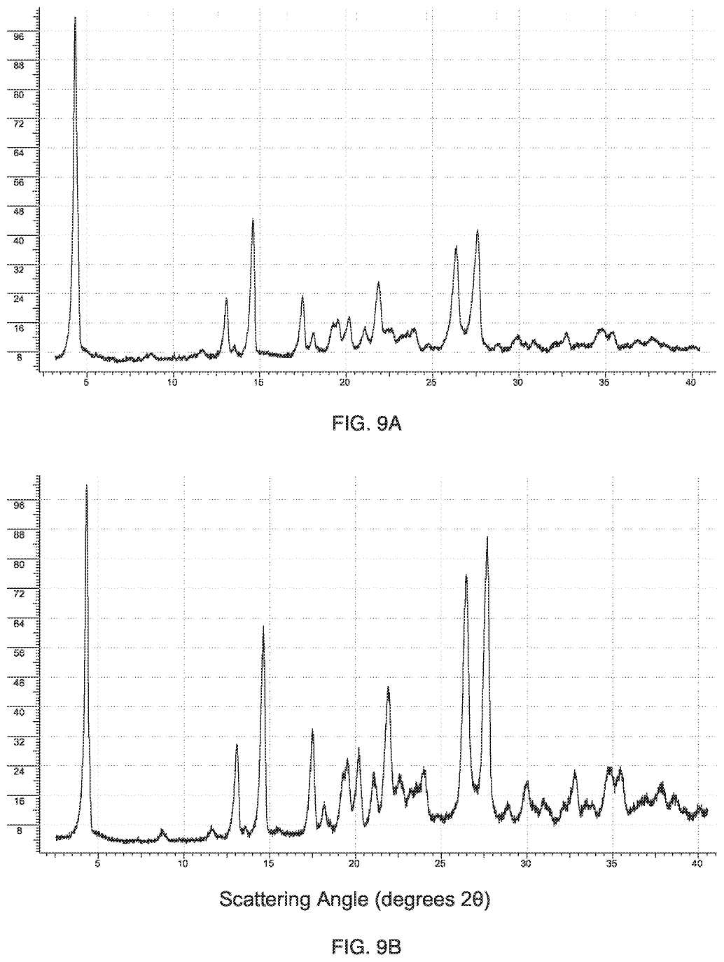

This example discloses the first preparation of the L-theanine/protocatechuic acid cocrystal product. 0.301 g of protocatechuic acid (1.953 mmol) and 0.342 g of L-theanine (1.963 mmol) were weighed directly into the bowl of an agate mortar, and wetted with 70% isopropanol to form a moderately thick slurry. The slurry was thoroughly ground at the time of mixing, and then periodically re-ground until the contents were dry. The XRPD pattern of the cocrystal product is illustrated in FIG. 9A. The XRPD pattern of the cocrystal product was found not to be a simple superimposition of the XRPD patterns of the reactants, demonstrating the formation of an authentic cocrystal product.

A second preparation of the L-theanine/protocatechuic acid cocrystal product was performed for the purpose of conducting a solubility study. This time, 0.441 g of protocatechuic acid (2.861 mmol) and 0.504 g of L-theanine (2.893 mmol) were weighed directly into the bowl of an agate mortar, and wetted with 70% isopropanol to form a moderately thick slurry. Once again, the slurry was thoroughly ground at the time of mixing, and then periodically re-ground until the contents were dry. As with the preparation discussed in the above paragraph, the XRPD pattern of the resulting cocrystal product was found not to be a simple superimposition of the XRPD patterns of the reactants, demonstrating the formation of an authentic cocrystal product. This XRPD pattern of the cocrsytal product is illustrated in FIG. 9B.

The following procedure was used to determine the solubility of the L-theanine/protocatechuic acid cocrystal in water:
2.0 mL of distilled water was placed in a volumetric tube.
Aliquots of solid cocrystal were added sequentially, and observations of the dissolution process were made using a low-power laser so as to observe any Tyndall effects. Table D illustrates the recorded observations:

TABLE D
Observations of the dissolution process

| Mass of | Cumulative | |
|---|---|---|
| Aliquot | Amount | |
| added (mg) | added (mg) | Observation |
| 6 | 6 | Total dissolution of the added solid |
| 8 | 14 | Total dissolution of the added solid |
| 11 | 25 | Total dissolution of the added solid |
| 13 | 38 | Total dissolution of the added solid |
| 14 | 52 | Total dissolution of the added solid |
| 11 | 63 | Partial dissolution of the added solid |

Based on these results, it was hypothesized that the aqueous solubility of the L-theanine/protocatechuic acid cocrystal was at least 26 mg/mL.

### REFERENCES

1. Medzhitov, Ruslan. “Inflammation 2010: New Adventures of an Old Flame.” www.ncbi.nlm.nih.gov/pubmed/20303867.
2. Kumar, V, et al. Robbins Basic Pathology. Elsevier, 2018. Chapter 3
3. Arulselvan, Palanisamy, et al. “Role of Antioxidants and Natural Products in Inflammation.” Oxidative Medicine and Cellular Longevity, vol. 2016, 2016, pp. 1-15.
4. Tandon V. R., Verma S., Singh J., Mahajan A. Antioxidants and cardiovascular health. Journal of Medical Education & Research. 2005; 7(2):115-118.
5. “Particle Pollution (PM).” Air Quality Index (AQI) Basics, airnow.gov/index.cfm-action-aqibasics.particle.
6. Poyton R. O., Ball K. A., Castello P. R. Mitochondrial generation of free radicals and hypoxic signaling. Trends in Endocrinology and Metabolism. 2009; 20(7):332-340.
7. Reuter S., Gupta S. C., Chaturvedi M. M., Aggarwal B. B. Oxidative stress, inflammation, and cancer: how are they linked? Free Radical Biology and Medicine. 2010; 49(11):1603-1616.
8. Coussens L. M., Werb Z. Inflammation and cancer. Nature. 2002; 420(6917):860-867.
9. Hussain S. P., Hofseth L. J., Harris C. C. Radical causes of cancer. Nature Reviews Cancer. 2003; 3(4):276-285.
10. Federico A., Morgillo F., Tuccillo C., Ciardiello F., Loguercio C. Chronic inflammation and oxidative stress in human carcinogenesis. International Journal of Cancer. 2007; 121(11):2381-2386.
11. Hussain S. P., Harris C. C. Inflammation and cancer: an ancient link with novel potentials. International Journal of Cancer. 2007; 121(11):2373-2380.
12. “List of Inflammatory Diseases.” ProgesteroneTherapy.com, www.progesteronetherapy.com/list-of-inflammatory-deseases.html.
13. Milenković, Dejan, et al. “Free Radical Scavenging Potency of Dihydroxybenzoic Acids.” Journal of Chemistry, vol. 2017, 2017, pp. 1-9.
14. S. Z. Dziedzic and B. J. F. Hudson, “Polyhydroxy chalcones and flavanones as antioxidants for edible oils,” Food Chemistry, vol. 12, no. 3, pp. 205-212, 1983.
15. C. A. Rice-Evans, N. J. Miller, and G. Paganga, “Structure-antioxidant activity relationships of flavonoids and phenolic acids,” Free Radical Biology and Medicine, vol. 20, no. 7, pp. 933-956, 1996.
16. J. M. Mayer, “Proton-coupled electron transfer: a reaction chemist's view,” Annual Review of Physical Chemistry, vol. 55, pp. 363-390, 2004.
17. M. H. V. Huynh and T. J. Meyer, “Proton-coupled electron transfer,” Chemical Reviews, vol. 107, no. 11, pp. 5004-5064, 2007.
18. Z. S. Marković, J. M. D. Marković, and Ć. B. Doličanin, “Mechanistic pathways for the reaction of quercetin with hydroperoxy radical,” Theoretical Chemistry Accounts, vol. 127, no. 1, pp. 69-80, 2010.
19. S. G. Chiodo, M. Leopoldini, N. Russo, and M. Toscano, “The inactivation of lipid peroxide radical by quercetin. A theoretical insight,” Physical Chemistry Chemical Physics, vol. 12, no. 27, pp. 7662-7670, 2010.
20. G. Litwinienko and K. U. Ingold, “Solvent effects on the rates and mechanisms of reaction of phenols with free radicals,” Accounts of Chemical Research, vol. 40, no. 3, pp. 222-230, 2007.
21. J. S. Wright, E. R. Johnson, and G. A. DiLabio, “Predicting the activity of phenolic antioxidants: theoretical method, analysis of substituent effects, and application to major families of antioxidants,” Journal of the American Chemical Society, vol. 123, no. 6, pp. 1173-1183, 2001.
22. E. Klein and V. Lukeš, “DFT/B3LYP study of the substituent effect on the reaction enthalpies of the individual steps of single electron transfer—proton transfer and sequential proton loss electron transfer mechanisms of phenols antioxidant action,” The Journal of Physical Chemistry A, vol. 110, no. 44, pp. 12312-12320, 2006.
23. G. A. DiLabio and K. U. Ingold, “A theoretical study of the iminoxyl/oxime self-exchange reaction. A five-center, cyclic proton-coupled electron transfer,” Journal of the American Chemical Society, vol. 127, no. 18, pp. 6693-6699, 2005.
24. L-Theanine Monograph http://www.chiro.org/nutrition/FULL/L-Theanine_Monograph.pdf
25. Unno T, Suzuki Y, Kakuda T, et al. Metabolism of theanine, a gamma-glutamylethylamide, in rats. J Agric Food Chem 1999; 47:1593-1596.
26. Wang, Dongxu, et al. “Protective Effect and Mechanism of Theanine on Lipopolysaccharide-Induced Inflammation and Acute Liver Injury in Mice.” Journal of Agricultural and Food Chemistry, vol. 66, no. 29, 2018, pp. 7674-7683.
27. Pérez-Vargas, Je, et al. “l-Theanine Prevents Carbon Tetrachloride-Induced Liver Fibrosis via Inhibition of Nuclear Factor KB and down-Regulation of Transforming Growth Factor β and Connective Tissue Growth Factor.” Human & Experimental Toxicology, vol. 35, no. 2, 2015, pp. 135-146.
28. Tsai, Wen-Hsin, et al. “l-Theanine Inhibits Proinflammatory PKC/ERK/ICAM-1/IL-33 Signaling, Apoptosis, and Autophagy Formation in Substance P-Induced Hyperactive Bladder in Rats.” Neurourology and Urodynamics, vol. 36, no. 2, 2016, pp. 297-307.
29. Yokozawa, Takako, and Erbo Dong. “Influence of Green Tea and Its Three Major Components upon Low-Density Lipoprotein Oxidation.” Experimental and Toxicologic Pathology, vol. 49, no. 5, 1997, pp. 329-335.
30. Li, Guilan, et al. “l-Theanine Prevents Alcoholic Liver Injury through Enhancing the Antioxidant Capability of Hepatocytes.” Food and Chemical Toxicology, vol. 50, no. 2, 2012, pp. 363-372.
31. Sugiyama, Tomomi, and Yasuyuki Sadzuka. “Theanine, a Specific Glutamate Derivative in Green Tea, Reduces the Adverse Reactions of Doxorubicin by Changing the Glutathione Level.” Cancer Letters, vol. 212, no. 2, 2004, pp. 177-184.
32. Neely, Kimberly A., and Thomas W. Gardner. “Ocular Neovascularization.” The American Journal of Pathology, vol. 153, no. 3, 1998, pp. 665-670.
33. Saito, Y., et al. “Effects of L-Theanine on Oxygen-Induced Retinal Neovascularization in the Neonatal Rat Model.” Investigative Ophthalmology & Visual Science, The Association for Research in Vision and Ophthalmology, 14 May 2008, iovs.arvojournals.org/article.aspx-articleid-2378047.
34. Hirooka, K., et al. “Theanine Provides Neuroprotective Effects of Retinal Ganglion Cells in a Rat Model of Chronic Glaucoma.” Investigative Ophthalmology & Visual Science, The Association for Research in Vision and Ophthalmology, 1 May 2003, ivos.arvojournals.org/article.aspx-articleid-2411898.
35. Liu, Jiaqi, et al. “Inflammation and Inflammatory Cells in Myocardial Infarction and Reperfusion Injury: A Double-Edged Sword,” Clinical Medicine Insights: Cardiology, vol. 10, 2016.
36. Siamwala, Jamila H., et al. “l-Theanine Promotes Nitric Oxide Production in Endothelial Cells through ENOS Phosphorylation.” The Journal of Nutritional Biochemistry, vol. 24, no. 3, 2013, pp. 595-605.
37. Yoto, Ai, et al. “Effects of L-Theanine or Caffeine Intake on Changes in Blood Pressure under Physical and Psychological Stresses.” Journal of Physiological Anthropology, vol. 31, no. 1, 2012, p. 28.
38. Kimura, Kenta, et al. “l-Theanine Reduces Psychological and Physiological Stress Responses.” Biological Psychology, vol. 74, no. 1, 2007, pp. 39-45.
39. Juneja, et al. “L-Theanine-a Unique Amino Acid of Green Tea and Its Relaxation Effect in Humans, Trends in Food Science & Technology.” DeepDyve, Elsevier, 1 Jun. 1999, www.deepdyve.com/lp/elsevier/l-theanine-a-unique-amino-acid-of-green-tea-and-its-relaxation-effect-icbzcGJZRi.
40. Nobre, A C, et al. “L-Theanine, a Natural Constituent in Tea, and Its Effect on Mental State.” Asia Pacific Journal of Clinical Nutrition., U.S. National Library of Medicine, www.ncbi.nlm.nih.gov/pubmed/18296328.
41. Nathan, Pradeep J., et al. “The Neuropharmacology of L-Theanine(N-Ethyl-L-Glutamine).” Journal of Herbal Pharmacotherapy, vol. 6, no. 2, 2006, pp. 21-30.
42. Yokogoshi, Hidehiko, et al. “Effect of Theanine, r-Glutamylethylamide, on Brain Monoamines and Striatal Dopamine Release in Conscious Rats.” SpringerLink, Springer, Dordrecht, link.springer.com/article/10.1023/A%3A1022490806093.
43. Ali, M., et al. “A Potent Thromboxane Formation Inhibitor in Green Tea Leaves.” Prostaglandins, Leukotrienes and Essential Fatty Acids, vol. 40, no. 4, 1990, pp. 281-283.
44. Li, Guilan, et al. “The Component of Green Tea, L-Theanine Protects Human Hepatic L02 Cells from Hydrogen Peroxide-Induced Apoptosis.” European Food Research and Technology, vol. 233, no. 3, 2011,pp. 427-435.
45. Kakkar, Sahil, and Souravh Bais. “A Review on Protocatechuic Acid and Its Pharmacological Potential.” ISRN Pharmacology, vol. 2014, 2014, pp. 1-9.
46. Semaming, Yoswaris, et al. “Pharmacological Properties of Protocatechuic Acid and Its Potential Roles as Complementary Medicine.” Evidence-Based Complementary and Alternative Medicine, vol. 2015, 2015, pp. 1-11.
47. W. L. Lin, Y. J. Hsieh, F. P. Chou, C. J. Wang, M. T. Cheng, and T. H. Tseng, “Hibiscus protocatechuic acid inhibits lipopolysaccharide-induced rat hepatic damage,” Archives of Toxicology, vol. 77, no. 1, pp. 42-47, 2003.
48. L. A. Pacheco-Palencia, S. Mertens-Talcott, and S. T. Talcott, “Chemical composition, antioxidant properties, and thermal stability of a phytochemical enriched oil from Acai (Euterpe oleracea Mart),” Journal of Agricultural and Food Chemistry, vol. 56, no. 12, pp. 4631-4636, 2008.
49. P. Li, X. Q. Wang, H. Z. Wang, and Y. N. Wu, “High performance liquid chromatographic determination of phenolic acids in fruits and vegetables,” Biomedical and Environmental Sciences, vol. 6, no. 4, pp. 389-398, 1993.
50. Liu, C L; Wang, J M; Chu, C Y; Cheng, M T; Tseng, T H (2002). “In vivo protective effect of protocatechuic acid on tert-butyl hydroperoxide-induced rat hepatotoxicity”. Food Chem Toxicol. 40 (5): 635-41.
51. Delsignore, A; Romeo, F; Giaccio, M (1997). “Content of phenolic substances in basidiomycetes”. Mycological Research. 101 (5): 552-6.
52. Lee Y S, Kang Y H, Jung J Y, et al. (October 2008). “Protein glycation inhibitors from the fruiting body of Phellinus linteus”. Biological & Pharmaceutical Bulletin. 31 (10): 1968-72.
53 . “3,4-Dihydroxybenzaldehyde.” National Center for Biotechnology Information. PubChem Compound Database, U.S. National Library of Medicine, pubchem.ncbi.nlm.nih.gov/compound/3,4-Dihydroxybenzaldehyde.
54. Chemical Land21, South Korea. http://chemicalland21.lookchem.com
55. Journal of Pharmacology and Experimental Therapeutics. Vol. 196, Pg. 478, 1976.
56. Pietta, P. G.; Simonetti, P.; Gardana, C.; Brusamolino, A.; Morazzoni, P.; Bombardelli, E. (1998). “Catechin metabolites after intake of green tea infusions”. BioFactors. 8 (1-2): 111-118.
57. Z. Sroka and W. Cisowski, “Hydrogen peroxide scavenging, antioxidant and anti-radical activity of some phenolic acids,” Food and Chemical Toxicology, vol. 41, no. 6, pp. 753-758, 2003.
58. R. Masella, R. Vari, M. D'Archivio et al., “Extra virgin olive oil biophenols inhibit cell-mediated oxidation of LDL by increasing the mRNA transcription of glutathione-related enzymes,” Journal of Nutrition, vol. 134, no. 4, pp. 785-791, 2004.
59. R. Vari, M. D'Archivio, C. Filesi et al., “Protocatechuic acid induces antioxidant/detoxifying enzyme expression through JNK-mediated Nrf2 activation in murine macrophages,” Journal of Nutritional Biochemistry, vol. 22, no. 5, pp. 409-417, 2011.
60. A. Tarozzi, F. Morroni, S. Hrelia et al., “Neuroprotective effects of anthocyanins and their in vivo metabolites in SH-SY5Y cells,” Neuroscience Letters, vol. 424, no. 1, pp. 36-40, 2007.
61. G. F. Shi, L. J. An, B. Jiang, S. Guan, and Y. M. Bao, “Alpinia protocatechuic acid protects against oxidative damage in vitro and reduces oxidative stress in vivo,” Neuroscience Letters, vol. 403, no. 3, pp. 206-210, 2006.
62. T.-H. Chou, H. Y. Ding, R. J. Lin, J. Y. Liang, and C.-H. Liang, “Inhibition of melanogenesis and oxidation by protocatechuic acid from Origanum vulgare (Oregano),” Journal of Natural Products, vol. 73, no. 11, pp. 1767-1774, 2010.
63. Y. Semaming, S. Kumfu, P. Pannangpetch, S. C. Chattipakorn, and N. Chattipakorn, “Protocatechuic acid exerts a cardioprotective effect in type 1 diabetic rats,” Journal of Endocrinology, vol. 223, no. 1, pp. 13-23, 2014.
64. A. R. Borate, A. A. Suralkar, S. S. Birje, P. V. Malusare, and P. A. Bangale, “Antihyperlipidemic effect of protocatechuic acid in fructose induced hyperlipidemia in rats,” International Journal of Pharma and Bio Sciences, vol. 2, no. 4, p. 456, 2011.
65. S. W. Min, S. N. Ryu, and D. H. Kim, “Anti-inflammatory effects of black rice, cyanidin-3-O-β-d-glycoside, and its metabolites, cyanidin and protocatechuic acid,” International Immunopharmacology, vol. 10, no. 8, pp. 959-966, 2010.
66. J. J. Yan, J. S. Jung, Y. J. Hong et al., “Protective effect of protocatechuic acid isopropyl ester against murine models of sepsis: inhibition of TNF-alpha and nitric oxide production and augmentation of IL-10,” Biological and Pharmaceutical Bulletin, vol. 27, no. 12, pp. 2024-2027, 2004.
67. D. Wang, X. Wei, X. Yan, T. Jin, and W. Ling, “Protocatechuic acid, a metabolite of anthocyanins, inhibits monocyte adhesion and reduces atherosclerosis in apolipoprotein E-deficient mice,” Journal of Agricultural and Food Chemistry, vol. 58, no. 24, pp. 12722-12728, 2010.
68. Lin, Chia-Yu, et al. “Anticoagulatory, Antiinflammatory, and Antioxidative Effects of Protocatechuic Acid in Diabetic Mice.” Journal of Agricultural and Food Chemistry, vol. 57, no. 15, 2009, pp. 6661-6667.
69. Cesari, M., Penninx, B. W., Newman, A. B., Kritchevsky, S. B., Nicklas, B. J., Sutton-Tyrrell, K., . . . Pahor, M. (2003). Inflammatory Markers and Onset of Cardiovascular Events. Circulation, 108(19), 2317-2322.
70. Gutierrez-Zetina, S., Gonzalez-Manzano, S., Perez-Alonso, J., Gonzalez-Paramas, A., & Santos-Buelga, C. (2019). Preparation and Characterization of Protocatechuic Acid Sulfates. Molecules, 24(2), 307.
71. Amin, H. P., Czank, C., Raheem, S., Zhang, Q., Botting, N. P., Cassidy, A., & Kay, C. D. (2015). Anthocyanins and their physiologically relevant metabolites alter the expression of IL-6 and VCAM-1 in CD4OL and oxidized LDL challenged vascular endothelial cells. Molecular Nutrition & Food Research, 59(6), 1095-1106.
72. J. R. Jones, C. Barrick, K. A. Kim et al., “Deletion of PPARγ in adipose tissues of mice protects against high fat diet-induced obesity and insulin resistance,” Proceedings of the National Academy of Sciences of the United States of America, vol. 102, no. 17, pp. 6207-6212, 2005.
73. B. Scazzocchio, R. Vari, C. Filesi et al., “Cyanidin-3-O-β-glucoside and protocatechuic acid exert insulin-like effects by upregulating PPARγ activity in human omental adipocytes,” Diabetes, vol. 60, no. 9, pp. 2234-2244, 2011.
74. R. Harini and K. V. Pugalendi, “Antihyperglycemic effect of protocatechuic acid on streptozotocin-diabetic rats,” Journal of Basic and Clinical Physiology and Pharmacology, vol. 21, no. 1, pp. 79-91, 2010.
75. Bhattacharjee, et al. “Protocatechuic Acid, a Phenolic from Sansevieria Roxburghiana Leaves, Suppresses Diabetic Cardiomyopathy via Stimulating Glucose Metabolism, Ameliorating Oxidative Stress, and Inhibiting Inflammation.”Frontiers, Frontiers, 19 Apr. 2017, www.frontiersin.org/articles/10.3389/fphar.2017.00251/full.
76. B. L. Queen and T. O. Tollefsbol, “Polyphenols and aging,” Current Aging Science, vol. 3, no. 1, pp. 34-42, 2010.
77. M. D'Archivio, C. Santangelo, B. Scazzocchioetal. “Modulatory effects of polyphenols on apoptosis induction: relevance for cancer prevention,” International Journal of Molecular Sciences, vol. 9, no. 3, pp. 213-228, 2008.
78. C. Giovannini, B. Scazzocchio, R. Vari, C. Santangelo, M. D'Archivio, and R. Masella, “Apoptosis in cancer and atherosclerosis: polyphenol activities,” Annali dell'Istituto Superiore di Sanita, vol. 43, no. 4, pp. 406-416, 2007.
79. J. Zhou-Stache, R. Buettner, G. Artmann, C. Mittermayer, and A. K. Bosserhoff, “Inhibition of TNF-α induced cell death in human umbilical vein endothelial cells and Jurkat cells by protocatechuic acid,”Medical and Biological Engineering and Computing, vol. 40, no. 6, pp. 698-703, 2002.
80. S. Guan, B. Jiang, Y. M. Bao, and L. J. An, “Protocatechuic acid suppresses MPP⁺-induced mitochondrial dysfunction and apoptotic cell death in PC12 cells,” Food and Chemical Toxicol-ogy, vol. 44, no. 10,pp. 1659-1666, 2006.
81. S. Guan, D. Ge, T. Q. Liu, X. H. Ma, and Z. F. Cui, “Protocatechuic acid promotes cell proliferation and reduces basal apoptosis in cultured neural stem cells,” Toxicology in Vitro, vol. 23, no. 2, pp. 201-208, 2009.
82. S. Guan, Y. M. Bao, B. Jiang, and L. J. An, “Protective effect of protocatechuic acid from Alpinia oxyphylla on hydrogen peroxide-induced oxidative PC12 cell death,” European Journal of Pharmacology, vol. 538, no. 1-3, pp. 73-79, 2006.
83. H. N. Zhang, C. N. An, M. Xu, D.-A. Guo, M. Li, and X.-P. Pu, “Protocatechuic acid inhibits rat pheochromocytoma cell damage induced by a dopaminergic neurotoxin,” Biological and Pharmaceutical Bulletin, vol. 32, no. 11, pp. 1866-1869, 2009.
84. T. H. Tseng, C. J. Wang, E. S. Kao, and H. Y. Chu, “Hibiscus protocatechuic acid protects against oxidative damage induced by tert-butylhydroperoxide in rat primary hepatocytes,” Chemico-Biological Interactions, vol. 101, no. 2, pp. 137-148, 1996.
85. C. Li, W. Jiang, H. Zhu, and J. Hou, “Antifibrotic effects of protocatechuic aldehyde on experimental liver fibrosis,” Pharmaceutical Biology, vol. 50, no. 4, pp. 413-419, 2012.
86. Chen, S.-Q., Wang, Z.-S., Ma, Y.-X., Zhang, W., Lu, J.-L., Liang, Y.-R., & Zheng, X.-Q. (2018). Neuroprotective Effects and Mechanisms of Tea Bioactive Components in Neurodegenerative Diseases. Molecules, 23(3), 512.
87. Tobaben S., Grohm J., Seiler A., Conrad M Plesnila N Culmsee C. Bid-mediated mitochondrial damage is a key mechanism in glutamate-induced oxidative stress and AIF-dependent cell death in immortalized HT-22 hippocampal neurons. Cell. Death Differ. 2011; 18:282-292.
88. Nozawa A., Umezawa K., Kobayashi K., Kawahara M., Muramoto K., Kakuda T., Kuroda Y. Theanine, a major flavorous amino acid in green tea leaves, inhibits glutamate-induced neurotoxicity on cultured rat cerebral cortical neurons. Soc. Neurosci. Abstr. 1998; 24:978.
89. Tani H., Dulla C. G., Farzampour Z., Taylor-Weiner A., Huguenard J. R., Reimer R. J. A local glutamate-glutamine cycle sustains synaptic excitatory transmitter release. Neuron. 2014; 81:888-900.
90. Kakuda T., Hinoi E., Abe A., Nozawa A., Ogura M., Yoneda Y. Theanine, an ingredient of green tea, inhibits [3H]glutamine transport in neurons and astroglia in rat brain. J. Neurosci. Res. 2008; 86:1846-1856.
91. Ogura M., Kakuda T., Takarada T., Nakamichi N., Fukumori R., Kim Y.H., Hinoi E., Yoneda Y. Promotion of both proliferation and neuronal differentiation in pluripotent P19 cells with stable overexpression of the glutamine transporter slc38a1. PLoS ONE. 2012; 7:e48270.
92. Takarada T., Ogura M., Nakamichi N., Kakuda T., Nakazato R., Kokubo H., Ikeno S., Nakamura S., Kutsukake T., Hinoi E., et al. Upregulation of slc38a1 gene along with promotion of neurosphere growth and subsequent neuronal specification in undifferentiated neural progenitor cells exposed to theanine. Neurochem. Res. 2016; 41:5-15.
93. Krzysztoforska, Kinga, et al. “Pharmacological Effects of Protocatechuic Acid and Its Therapeutic Potential in Neurodegenerative Diseases: Review on the Basis of in Vitro and in Vivo Studies in Rodents and Humans.” Nutritional Neuroscience, vol. 22, no. 2,2017, pp. 72-82.
94. Li J, O W, Li W, Jiang Z G, Ghanbari H. Oxidative stress and neurodegenerative disorders. Int J Mol Sci 2013; 14(12):24438-75.
95. Miller E, Morel A, Saso L, Saluk J. Isoprostanes and neuroprostanes as biomarkers of oxidative stress in neurodegenerative diseases. Oxid Med Cell Longev 2014; 2014:1-10.
96. Kim G H, Kim J E, Rhie S J, Yoon S. The role of oxidative stress in neurodegenerative diseases. Exp Neurobiol 2015; 24(4):325-40.
97. Thakare V N, Dhakane V D, Patel B M. Attenuation of acute restraint stress-induced depressive like behavior and hippocampal alterations with protocatechuic acid treatment in mice. Metab Brain Dis 2016; 32(2):401-13.
98. Galano A, Pérez-González A. On the free radical scavenging mechanism of protocatechuic acid, regeneration of the catechol group in aqueous solution. Theor Chem Acc 2012; 131(9):1-13.
99. Yin X, Zhang X, Lv C, Li C, Yu Y, Wang X, et al. Protocatechuic acid ameliorates neurocognitive functions impairment induced by chronic intermittent hypoxia. Sci Rep 2015; 5:14507.
100. Wu Y X, Wu T Y, Xu B B, Xu X Y, Chen H G, Li X Y,et al. Protocatechuic acid inhibits osteoclast differentiation and stimulates apoptosis in mature osteoclasts. Biomed Pharmacother. 2016; 82:399-405.
101. Prasad, K. N. (2017). Oxidative Stress, Pro-Inflammatory Cytokines, and Antioxidants Regulate Expression Levels of MicroRNAs in Parkinson's Disease. Current Aging Science, 10(3):177-184.

Each reference cited above is hereby incorporated in its entirety as if fully set forth herein.

1-33. (canceled)

34. A composition comprising protocatechuic acid and L-theanine.

35. The composition of claim 34, wherein protocatechuic acid and L-theanine are in the form of a cocrystal.

36. The composition of claim 34, wherein the wt. % of protocatechuic acid in the composition is between about 40% and about 50%.

37. The composition of claim 34, wherein the wt. % of L-theanine in the composition is between about 50% and about 60%.

38. A dosage form comprising the composition of claim 34.

39. The dosage form of claim 38, wherein protocatechuic acid and L-theanine are in the form of a cocrystal.

40. The dosage form of claim 38, wherein the dosage form is an oral disintegrating tablet or an amount of powder.

41. A vitamin or fruit juice drink comprising the dosage form of claim 38, wherein the drink optionally comprises electrolytes.

42. The dosage form of claim 38, wherein the amount of protocatechuic acid in the dosage form is between about 25 mg and about 600 mg.

43. The dosage form of claim 38, wherein the amount of L-theanine in the dosage form is between about 300 mg and about 550 mg.

44. A formulation comprising: protocatechuic acid and L-theanine; or protocatechuic aldehyde and L-theanine.

45. The formulation of claim 44, wherein the formulation comprising protocatechuic acid and L-theanine is a cocrystal formulation.

46. The formulation of claim 44, wherein the formulation comprising protocatechuic aldehyde and L-theanine is water soluble.

47. The formulation of claim 46, wherein the formulation crosses the blood-brain barrier.

48. The formulation of claim 45, wherein the cocrystal protocatechuic acid and L-theanine formulation is water soluble.

49. The formulation of claim 48, wherein the formulation crosses the blood-brain barrier.

50. The formulation of claim 44, wherein the formulation comprising protocatechuic aldehyde and L-theanine is in the form of an oral disintegrating tablet or an amount powder.

51. The formulation of claim 45, wherein the cocrystal protocatechuic acid and L-theanine formulation is in the form of an oral disintegrating tablet or an amount powder.

52. The formulation of claim 44, wherein the formulation comprising protocatechuic aldehyde and L-theanine is a sublingual formulation.

53. The formulation of claim 45, wherein the cocrystal protocatechuic acid and L-theanine formulation is a sublingual formulation.
